(19) United States
(12) Patent Application Publication
(10) Pub. No.: US 2012/0196828 A1
(43) Pub. Date: Aug. 2, 2012

(54) SENSITIZATION OF CANCER CELLS TO TREATMENT

(75) Inventor: Stephen MARCUS, Weston, FL (US)

(73) Assignee: ParinGenix, Inc., Weston, FL (US)

(21) Appl. No.: 13/363,185

(22) Filed: Jan. 31, 2012

Related U.S. Application Data
(60) Provisional application No. 61/438,574, filed on Feb. 1, 2011.
(60) Provisional application No. 61/493,320, filed on Jun. 3, 2011.
(60) Provisional application No. 61/583,795, filed on Jan. 6, 2012.

(51) Int. Cl. A61K 31/727; A61P 35/00; A61K 31/7068

(52) U.S. Cl. 514/49; 514/56

(57) ABSTRACT

The present application provides methods for the treatment of cancer, comprising administering substantially non-anticoagulant 2-O, 3-O desulfated heparin to patients suffering from cancer that is, or can become resistant to, cancer treatment, such as chemotherapy, targeted cancer therapy, or radiation therapy. The compositions can be administered to sensitize, or to reverse resistance to, cancer treatment, and can be administered alone or in combination with cancer treatment to subjects with solid tumors including, but not limited to, pancreatic, breast, renal, colorectal, gastric, or esophageal cancer, and subjects with hematologic malignancies, including but not limited to leukemia and lymphoma.

### 1. CROSS REFERENCE TO RELATED APPLICATIONS

This application claims the benefit under 35 U.S.C. §119(e) of provisional application No. 61/438,574, filed Feb. 1, 2011, provisional application No. 61/493,320, filed Jun. 3, 2011, and provisional application No. 61/583,795, filed Jan. 6, 2012, the contents of all of which are incorporated herein in their entireties by reference thereto.

### 2. BACKGROUND

Resistance to cancer therapies—including therapies that are nonspecifically cytotoxic to dividing cells and therapies that are more specific for targets in neoplastic cells—is a continuing problem in oncology.

Studies on the ability of cancer cells to survive treatment with broadly cytotoxic agents, radiation, and/or targeted therapies have led to the appreciation that many such treatments are only moderately effective and can become less effective as tumors develop resistance. One mechanism by which tumors may develop resistance to anti-cancer treatments is autophagy, a self-catabolic process that maintains intracellular homeostasis and prolongs cell survival during periods of stress. Chen, N., et al., 2011, Cancer Biology & Therapy 11(2): 157-168. In studies of resistant cancer cells, autophagy appears to be upregulated along with decreased activation of apoptosis, or programmed cell death. Kang, R., et al., 2010, Cell Death and Differentiation, 17:666-676; Kang, R., et al., 2011, Autophagy, 7(1): 91-93.

The failure of first-line and subsequent therapies and development of treatment-resistant tumors is a significant problem in the care and management of patients suffering from cancer. New ways of maintaining or increasing the efficacy of such treatments are urgently needed, including methods of reducing or preventing autophagy-mediated de-sensitization or resistance to cancer treatment.

### 3. SUMMARY

It has been discovered that substantially non-anticoagulant 2-O, 3-O desulfated heparin (ODSH) improves and/or sustains the efficacy over time of a cytotoxic regimen in a standard tumor xenograft animal model of human pancreatic cancer, a cancer that often exhibits and/or develops resistance to treatment. ODSH, and compositions thereof, are therefore useful in the treatment of cancers that are, or are likely to become, resistant to cancer treatments.

In an aspect, the present disclosure provides methods of treating solid tumors or hematologic malignancies that are, or can become, resistant to cancer treatments, such as chemotherapy, targeted cancer therapy, and radiation therapy. Generally, the methods of treatment involve administering ODSH to a subject diagnosed with a cancer that is, or can become, resistant to cancer treatment. Subjects can be treatment naïve, i.e. never before treated with a cancer treatment, or can previously have been treated with one or more cancer treatments. Subjects can have cancers that are resistant to cancer treatments, or cancers that likely to become resistant to cancer treatments.

ODSH, or compositions thereof, may be administered alone as a monotherapy, to subjects who have previously received cancer treatment or who have been diagnosed with cancer that is, or is likely to become, resistant to cancer treatment. ODSH may be administered for a specified period of time or continuously.

Alternatively, ODSH may be administered in combination with, or adjunctive to, a cancer treatment that is tumor-appropriate. When used in combination with, or adjunctive to, cancer treatment, ODSH can be administered prior to, concomitant with, or subsequent to cancer treatment, or any combination thereof. ODSH may be administered for a specified period of time or continuously.

In one aspect, the present disclosure provides a method of treating pancreatic cancer that involves administering ODSH to a subject diagnosed with pancreatic cancer that is, or may become, resistant to cancer treatment. ODSH may be administered alone as a monotherapy. Alternatively, ODSH may be administered in combination with, or adjunctive to, cancer treatment.

In some embodiments, ODSH is administered in combination with one or more chemotherapeutic agents. Specific non-limiting examples of chemotherapeutic agents which can be used alone or in combination include gemcitabine and nab-paclitaxel. ODSH can be administered as a continuous infusion, starting either concurrently with a chemotherapeutic agent or immediately thereafter. Optionally, a first “loading dose” of ODSH can be administered as a bolus immediately before or after a chemotherapeutic agent is administered. In a specific embodiment, ODSH is administered as a bolus immediately after chemotherapy with either gemcitabine or gemcitabine and nab-paclitaxel, followed by administration as a continuous intravenous infusion.

Many cancers, including cancers that show increased resistance to cancer treatments, show increased expression of High Mobility Group Box 1 protein, a DNA-binding protein that functions as a cytokine, and of one of the receptors to which it binds, the Receptor for Advanced Glycation End products (RAGE). These proteins have been implicated in the regulation of autophagy, a cellular stress response, which is also associated with increased resistance of cancer cells to cytotoxic treatment. 2-O, 3-O desulfated heparin (ODSH) has been shown to disrupt the interaction between RAGE and HMGB1. Without intending to be bound by any theory of operation, it is believed that ODSH, by inhibiting the interaction of HMGB1 with RAGE, can curtail autophagy in cancer cells, thereby reducing or preventing their resistance to chemotherapy, targeted cancer therapy, and radiation therapy. Thus, ODSH is of particular utility in the treatment of cancers that are, or can become, resistant to treatment via autophagy.

Cancers that can become resistant to cancer treatment include cancers known to develop resistance, e.g. based on experimental or clinical data, as well as cancers in which the genes encoding HMGB1 or RAGE are expressed at higher levels than in non-cancerous tissue. Described herein are methods using compositions of ODSH for treating cancers, including “solid tumors,” including, but not limited to breast, renal, brain, prostate, melanoma, gastric, liver, nasopharyngeal, head and neck, esophagus, ovarian and colorectal cancers, and hematologic malignancies, including but not limited to leukemia, lymphoma and myeloma.

In one aspect, the present disclosure provides a method of treating breast cancer that involves administering ODSH to a subject diagnosed with breast cancer that is, or may become, resistant to cancer treatment. ODSH may be administered alone as a monotherapy. Alternatively, ODSH may be administered in combination with, or adjunctive to, cancer treatment. In some embodiments, the cancer treatment is chemotherapy. In some embodiments, ODSH is administered in combination with a targeted agent, for example, an anti-HER2 monoclonal antibody.

In one aspect, the present disclosure provides a method of treating renal cancer that involves administering ODSH to a subject diagnosed with renal cancer that is, or may become, resistant to cancer treatment. ODSH may be administered alone as a monotherapy. Alternatively, ODSH may be administered in combination with, or adjunctive to, cancer treatment. In some embodiments, ODSH may be administered in combination with targeted cancer therapy, for example, a therapy that inhibits the mammalian target of rapamycin (mTOR).

In one aspect, the present disclosure provides a method of treating colorectal cancer that involves administering ODSH to a subject diagnosed with colorectal cancer that is, or may become, resistant to cancer treatment. ODSH may be administered alone as a monotherapy. Alternatively, ODSH may be administered in combination with, or adjunctive to, cancer treatment. In some embodiments, ODSH may be administered in combination with, or adjunctive to, chemotherapeutic agents, for example, 5-fluorouracil, 5-fluorouracil prodrugs, such as Xeloda®, irinotecan, leucovorin, and/or oxaliplatin.

In one aspect, the present disclosure provides a method of treating gastric cancer that involves administering ODSH to a subject diagnosed with gastric cancer that is, or may become, resistant to cancer treatment. ODSH may be administered alone as a monotherapy. Alternatively, ODSH may be administered in combination with, or adjunctive to, cancer treatment. In some embodiments, the cancer treatment is chemotherapy and may be administered in combination with, or adjunctive to, chemotherapeutic agents, for example, docetaxel, cisplatin, and/or 5-Fluorouracil. In some embodiments, ODSH is administered in combination with a targeted agent, for example, an anti-HER2 monoclonal antibody or an anti-VEGF monoclonal antibody.

In one aspect, the present disclosure provides a method of treating esophageal cancer that involves administering ODSH to a subject diagnosed with esophageal cancer that is, or may become, resistant to cancer treatment. ODSH may be administered alone as a monotherapy. Alternatively, ODSH may be administered in combination with, or adjunctive to, cancer treatment. In some embodiments, the cancer treatment is chemotherapy and may be administered in combination with, or adjunctive to, chemotherapeutic agents, for example, docetaxel, cisplatin, and/or 5-Fluorouracil. In some embodiments, ODSH is administered in combination with, or adjunctive to, radiation therapy. In some embodiments, ODSH is administered in combination with a targeted agent, for example, an anti-HER2 monoclonal antibody or an anti-VEGF monoclonal antibody.

The present disclosure provides pharmaceutical compositions and unit dosage forms of ODSH, suitable for use in the methods described above, either as monotherapy or in combination with chemotherapy, radiation therapy, or targeted cancer therapy. The pharmaceutical compositions may be prepared for parenteral administration, such as intravenous or subcutaneous administration. For intravenous administration, pharmaceutical compositions can be formulated for administration as a bolus or as a continuous infusion, at doses of ODSH ranging from about 0.1 mg/kg/hr to about 2.5 mg/kg/hr for infusions, and from about 1 mg/kg to about 25 mg/kg for bolus doses. For subcutaneous administration, pharmaceutical compositions can be formulated for administration at doses ranging from about 25 mg to about 400 mg, in volumes of 2.0 mL of less per injection site.

### 4. BRIEF DESCRIPTION OF THE DRAWINGS

FIG. 1 provides a graph illustrating the effect on tumor weight of 8 different regimens: vehicle control (Group 1, ), ODSH alone (Group 2, ◯), oxaliplatin/gemcitabine/nab-paclitaxel (Group 3, ▪), gemcitabine alone (Group 4, □), oxaliplatin/gemcitabine/nab-paclitaxel with ODSH (Group 5, ▴), gemcitabine with ODSH (Group 6, Δ), oxaliplatin/gemcitabine (Group 7, x), and oxaliplatin/gemcitabine with ODSH (Group 8, ⋆) as described further in Example 1 and at Table 1;

FIG. 2 provides a graph illustrating the effect on tumor weight of a subset of the regimens shown in FIG. 1: vehicle control (Group 1, ), ODSH alone (Group 2, ◯), gemcitabine alone (Group 4, □) and gemcitabine with ODSH (Group 6, Δ); and

FIG. 3 provides a graph illustrating the effect on body weight of 8 different regimens: vehicle control (Group 1, ), ODSH alone (Group 2, ◯), oxaliplatin/gemcitabine/nab-paclitaxel (Group 3, ▪), gemcitabine alone (Group 4, □), oxaliplatin/gemcitabine/nab-paclitaxel with ODSH (Group 5, ▴), gemcitabine with ODSH (Group 6, Δ), oxaliplatin/gemcitabine (Group 7, x), and oxaliplatin/gemcitabine with ODSH (Group 8, ⋆) as described further in Example 1 and at Table 1.

### 5. DETAILED DESCRIPTION

5.1. Overview

It has now been discovered that adjunctive administration of a substantially non-anticoagulant 2-O, 3-O-desulfated heparin composition (ODSH) with a tumor-appropriate cytotoxic regimen improves and/or sustains the efficacy over time of the cytotoxic regimen in a standard tumor xenograft animal model of human pancreatic cancer, improving clinical outcome. In particular, as further described in Example 1, adjunctive administration of ODSH with gemcitabine resulted in a statistically significant inhibition of tumor growth relative to treatments with the vehicle control alone. Furthermore, ODSH and gemcitabine had a synergistic effect when administered in combination, giving rise to a greater percent tumor growth inhibition than either compound administered alone.

5.2. Methods of Treatment

5.2.1. Treatment by Adjunctive Administration of ODSH

Thus, in a first aspect, methods of treating cancer are provided, comprising adjunctively administering ODSH and a tumor-appropriate cancer treatment to a subject in need of cancer treatment.

As used herein, cancer treatment includes therapy with a chemotherapeutic agent (chemotherapy), therapy with an agent acting on at least one target thought to play a role in cancer (targeted cancer therapy), or therapy with ionizing radiation (radiation therapy). Numerous chemotherapeutic agents, e.g. cytotoxic agents, and their uses in treating individual cancers are known in the art. Similarly, targeted cancer therapies developed to treat individual cancers are known in the art. These include monoclonal antibodies that target proteins known to play a role in a specific cancer or tumor being treated. One example of a monoclonal antibody is trastuzumab (HERCEPTIN®), an anti-HER2 monoclonal antibody that targets HER2-positive breast cancers. Other examples of targeted cancer therapies include inhibitors of mammalian Target Of Rapamycin (mTOR), a protein known to play a role in a number of cancers.

Subjects can be treatment-naïve, i.e. never before treated with a cancer treatment, or may previously have been treated with cancer treatment. In certain embodiments, subjects have cancers that are resistant to cancer treatments. In various embodiments, subjects have cancers that may become resistant to cancer treatments. Cancers that can become resistant to cancer treatment include cancers known to develop resistance, e.g. based on experimental or clinical data, as well as cancers in which the genes associated with resistance and/or desensitization to cancer treatment are expressed at higher levels than in non-cancer cells. The subject treated may be any animal, for example, a mammal, particularly a human.

ODSH is substantially non-anticoagulating. Accordingly, ODSH can be used in subjects in whom use of anti-coagulants is contra-indicated, and can generally be used at higher doses than heparin for treatments where anti-coagulation is not desired or needed. Furthermore, ODSH does not induce, and can also prevent, heparin-induced thrombocytopenia (HIT), a rare but very serious side-effect of heparin. As such, ODSH can be used in subjects who are at risk for HIT. See U.S. Pat. No. 7,468,358.

The phrases adjunctive administration of ODSH, adjunctively administering ODSH, administering ODSH in combination with, or adjunctive to, as used herein, are used interchangeably and mean administering ODSH as part of a treatment regimen that includes a cancer treatment. Adjunctive administration includes administration concurrently with, sequentially with, or separately from, administration of the cancer treatment. Administration is said to be sequential if ODSH is administered on the same day as cancer treatment, for example during the same patient visit, but not concurrently. Administration is said to separate if ODSH is administered on a different day from the day the subject receives cancer treatment but during an ongoing treatment regimen. When administered separately or sequentially, ODSH can be administered before, after, or both before and after cancer treatment.

ODSH can be administrated via the same or different route as the administered cancer treatment. Therapeutic regimens for adjunctive administration of ODSH with cancer treatment can include combinations of concurrent, sequential, and separate administration, for example, concurrent administration on certain days, and/or separate on other days, and/or sequential on yet other days.

In some embodiments, ODSH is administered parenterally. In certain embodiments, ODSH is administered intravenously, either as a bolus, as a continuous infusion, or as a bolus followed by continuous infusion.

ODSH is administered for a time and in an amount sufficient to provide a therapeutic benefit.

In various embodiments, ODSH is administered over a period of 2 weeks to indefinitely, a period of 2 weeks to 6 months, a period of 3 months to 5 years, a period of 6 months to 1 or 2 years, or the like. Optionally, ODSH administration can be repeated, for example, once daily, twice daily, every two days, every three days, every five days, once a week, once every two weeks, or once a month. Some treatment regimens may include a period of several weeks of regular ODSH administration followed by a period of rest, when no ODSH is administered. For example, a treatment regimen can include one, two, three, or more weeks of ODSH administration followed by one, two, three, or more weeks without ODSH administration. The repeated administration can be at the same dose or at a different dose. Administration of cancer treatment, e.g., chemotherapy, radiation therapy, targeted cancer therapy, can be carried out according to standard regimens, known to those skilled in the art.

ODSH is administered to the subject in an amount sufficient or effective to provide a therapeutic benefit. In the context of treating cancers that are or can become resistant to cancer treatment, a therapeutic benefit can be inferred, if one or more of the following is achieved: re-sensitizing resistant cancer cells to cancer treatment, preventing the development of resistance of cancer cells to cancer treatment, halting or slowing the growth of tumors, reducing the size and/or number of tumors within a patient, increasing life expectancy, and/or improving patient quality of life. A complete cure, while desirable, is not required for therapeutic benefit to exist.

In some contexts, a therapeutic benefit can be correlated with one or more surrogate end points, in accordance with the knowledge of one of ordinary skill in the art. By way of example and not limitation, reducing or preventing resistance of, or sensitizing, a subject's tumor to cancer treatment is indicative of therapeutic benefit, and can be measured in vivo or in vitro. Sensitization of cancer cells to cancer treatment can, for example, be measured in vitro by exposing cancer cells to a fixed dose of cancer treatment with or without ODSH and assaying for a reduction in cell viability or autophagosome formation (LC3 puntae staining). See Vazquez-Martin, A., et al., 2009, supra; Kang, R., 2010, Cell Death & Differentiation, supra.

The amount of ODSH administered will depend on various factors, including the nature and stage of the cancer being treated, the form, route, and site of administration, the therapeutic regimen (for example, whether a chemotherapeutic agent is used in addition to ODSH), the age and condition of the subject being treated. The appropriate dosage can be readily determined by a person of skill in the art. In practice, a physician will determine appropriate dosages to be used. This dosage can be repeated as often as appropriate. The amount and/or frequency of the dosage can be altered, increased, or reduced, depending on the subject's response and in accordance with standard clinical practice. The proper dosage and treatment regimen can be established by monitoring the progress of therapy using conventional techniques known to people skilled in the art.

Effective dosages can be estimated initially from in vitro assays or in vivo assays in animals. For example, an initial dose used in animals may be formulated to achieve a desired circulating blood or serum concentration of ODSH. Calculating dosages to achieve such circulating blood or serum concentrations taking into account bioavailability of ODSH is well within the capabilities of skilled artisans. Ordinarily skilled artisans can routinely adapt information derived from relevant animal models useful for testing the efficacy of compounds, to determine dosages suitable for human administration. See, e.g., Example 1 below for an animal model testing efficacy in a human pancreatic tumor xenograft mouse model. Further guidance can be found, for example, in Fingl & Woodbury, “General Principles” in Goodman and Gilman's The Pharmaceutical Basis of Therapeutics, Chapter 1, latest edition, Pagamon Press, and references cited therein.

In some embodiments, ODSH is administered at a dose or amount per kilogram of patient body weight ranging from about 1 mg/kg to about 25 mg/kg for bolus doses, and from about 0.1 mg/kg/hr to about 2.5 mg/kg/hr for infusions. In a specific embodiment, ODSH is administered as a bolus at a dose of about 4 mg/kg, optionally followed by an intravenous infusion of ODSH at a dose of about 0.375 mg/kg/hr for 48 hours. A bolus dose can be administered over less than a minute, about a minute, about 2 minutes, about 3 minutes, about 4 minutes, or about 5 minutes. For subcutaneous administration, ODSH can be administered at doses ranging from about 25 mg to about 400 mg, in volumes of 2.0 mL of less per injection site.

Pharmaceutical compositions of ODSH can be formulated in an amount that permits bolus intravenous administration and/or continuous intravenous infusion at such doses. In one embodiment, the pharmaceutical composition comprises ODSH in a sterile vial at a concentration of 50 mg/mL. When formulated for subcutaneous administration, pharmaceutical compositions can contain ODSH at a concentration ranging from 50 mg/ml to 350 mg/ml suitable for administration at doses ranging from about 25 to about 400 mg, in volumes of 2.0 mL or less per injection site.

Optionally, the methods of the present application can comprise a step of determining expression level of RAGE or HMGB1 in a tumor sample from a subject, prior to administration of ODSH.

5.2.2. Treatment by Sole Administration of ODSH

It has now also been found that sole administration of ODSH inhibits tumor growth to a similar extent as treatment with gemcitabine, a tumor-appropriate chemotherapeutic agent, in a standard tumor xenograft animal model of human pancreatic cancer, improving clinical outcome. See Example 1. Thus, in another aspect, methods of treating cancer are provided, comprising administering ODSH to a subject in need of cancer treatment.

Subjects can be treatment-naïve, i.e. never before treated with a cancer treatment, or may previously have been treated with cancer treatment. In certain embodiments, subjects have cancers that are resistant to cancer treatments. In various embodiments, subjects have cancers that may become resistant to cancer treatments. Cancers that can become resistant to cancer treatment include cancers known to develop resistance, e.g. based on experimental or clinical data, as well as cancers in which the genes encoding HMGB1 or RAGE are expressed at higher levels than in non-cancer cells. The subject treated may be any animal, for example, a mammal, particularly a human.

ODSH is substantially non-anticoagulating. Accordingly, ODSH can be used in subjects in whom use of anti-coagulants is contra-indicated, and can generally be used at higher doses than heparin for treatments where anti-coagulation is not desired or needed. Furthermore, ODSH does not induce, and can also prevent, heparin-induced thrombocytopenia (HIT), a rare but very serious side-effect of heparin. As such, ODSH can be used in subjects who are at risk for HIT. See U.S. Pat. No. 7,468,358.

In some embodiments, ODSH is administered parenterally. In certain embodiments, ODSH is administered intravenously, either as a bolus, as a continuous infusion, or as a bolus followed by continuous infusion.

ODSH is administered for a time and in an amount sufficient to provide a therapeutic benefit.

In various embodiments, ODSH is administered over a period of 2 weeks to indefinitely, a period of 2 weeks to 6 months, a period of 3 months to 5 years, a period of 6 months to 1 or 2 years, or the like. Optionally, ODSH administration can be repeated, for example, once daily, twice daily, every two days, every three days, every five days, once a week, once every two weeks, or once a month. Some treatment regimens may include a period of several weeks of regular ODSH administration followed by a period of rest, when no ODSH is administered. For example, a treatment regimen can include one, two, three, or more weeks of ODSH administration followed by one, two, three, or more weeks without ODSH administration. The repeated administration can be at the same dose or at a different dose.

ODSH is administered to the subject in an amount sufficient or effective to provide a therapeutic benefit. In the context of treating cancers that are or can become resistant to cancer treatment, a therapeutic benefit can be inferred if one or more of the following is achieved: re-sensitizing resistant cancer cells to cancer treatment, preventing the development of resistance of cancer cells to cancer treatment, halting or slowing the growth of tumors, reducing the size and/or number of tumors within a patient, increasing life expectancy, and/or improving patient quality of life. A complete cure, while desirable, is not required for therapeutic benefit to exist.

In some contexts, a therapeutic benefit can be correlated with one or more surrogate end points, in accordance with the knowledge of one of ordinary skill in the art. By way of example and not limitation, reducing or preventing resistance of, or sensitizing, a subject's tumor to cancer treatment is indicative of therapeutic benefit, and can be measured in vivo or in vitro. Sensitization of cancer cells to cancer treatment can, for example, be measured in vitro by exposing cancer cells to a fixed dose of cancer treatment with or without ODSH and assaying for a reduction in cell viability or autophagosome formation (LC3 puntae staining). See Vazquez-Martin, A., et al., 2009, supra; Kang, R., 2010, Cell Death & Differentiation, supra.

The amount of ODSH administered will depend on various factors, including the nature and stage of the cancer being treated, the form, route, and site of administration, the therapeutic regimen, the age and condition of the subject being treated. The appropriate dosage can be readily determined by a person of skill in the art. In practice, a physician will determine appropriate dosages to be used. This dosage can be repeated as often as appropriate. The amount and/or frequency of the dosage can be altered, increased, or reduced, depending on the subject's response and in accordance with standard clinical practice. The proper dosage and treatment regimen can be established by monitoring the progress of therapy using conventional techniques known to people skilled in the art.

Effective dosages can be estimated initially from in vitro assays or in vivo assays in animals. For example, an initial dose used in animals may be formulated to achieve a desired circulating blood or serum concentration of ODSH. Calculating dosages to achieve such circulating blood or serum concentrations taking into account bioavailability of ODSH is well within the capabilities of skilled artisans. Ordinarily skilled artisans can routinely adapt information derived from relevant animal models useful for testing the efficacy of compounds, to determine dosages suitable for human administration. See, e.g., Example 1 below for an animal model testing efficacy in a human pancreatic tumor xenograft mouse model. Further guidance can be found, for example, in Fingl & Woodbury, “General Principles” in Goodman and Gilman's The Pharmaceutical Basis of Therapeutics, Chapter 1, latest edition, Pagamon Press, and references cited therein.

In some embodiments, ODSH is administered at a dose or amount per kilogram of patient body weight ranging from about 1 mg/kg to about 25 mg/kg for bolus doses, and from about 0.1 mg/kg/hr to about 2.5 mg/kg/hr for infusions. In a specific embodiment, ODSH is administered as a bolus at a dose of about 4 mg/kg, optionally followed by an intravenous infusion of ODSH at a dose of about 0.375 mg/kg/hr for 48 hours. A bolus dose can be administered over less than a minute, about a minute, about 2 minutes, about 3 minutes, about 4 minutes, or about 5 minutes. For subcutaneous administration, ODSH can be administered at doses ranging from about 25 mg to about 400 mg, in volumes of 2.0 mL of less per injection site.

Pharmaceutical compositions of ODSH can be formulated in an amount that permits bolus intravenous administration and/or continuous intravenous infusion at such doses. In one embodiment, the pharmaceutical composition comprises ODSH in a sterile vial at a concentration of 50 mg/mL. When formulated for subcutaneous administration, pharmaceutical compositions can contain ODSH at a concentration ranging from 50 mg/ml to 350 mg/ml suitable for administration at doses ranging from about 25 to about 400 mg, in volumes of 2.0 mL or less per injection site.

Optionally, the methods of the present application can comprise a step of determining expression level of RAGE or HMGB1 in a tumor sample from a subject, prior to administration of ODSH.

5.3. Treatment of Pancreatic Cancer

Patients diagnosed with pancreatic cancer typically have a poor prognosis, in part because pancreatic cancer causes few symptoms until the disease has progressed to an advanced stage and is incurable with surgery. Additionally, pancreatic cancer is prone to developing resistance to cancer treatment. Applicant has discovered that ODSH can enhance responsiveness to standard therapy for the treatment of pancreatic cancer. As shown in Example 1, ODSH administered alone or adjunctive to appropriate cancer treatment, results in an inhibition of tumor growth in an accepted animal xenograft model of human pancreatic cancer. (See Tan, et al., 1986, Cancer Invest. 4(1): 15-23, describing the BxPC-3 xenograft model in athymic nude mice). Surprisingly, the tumor growth inhibition is increased by adjunctive administration of ODSH with a cancer treatment. As such, subjects suffering from pancreatic cancer are candidates for treatment with ODSH. Accordingly, in one aspect, the methods of the present application comprise treating pancreatic cancer by adjunctively administering ODSH with cancer treatment, as described above in Section 5.2.1, to a subject suffering from pancreatic cancer. Alternatively, the methods of the present application comprise treating cancer by administering ODSH alone, as described above in Section 5.2.2, to a subject suffering from pancreatic cancer.

ODSH can be administered to subjects with pancreatic cancer to prevent resistance to cancer treatment or re-sensitize cancers that have acquired resistance to such treatments. In some embodiments, ODSH is administered in combination with, or adjunctive to, one or more chemotherapeutic agents, such as gemcitabine, gemcitabine and one or more additional chemotherapeutic agent (e.g., gemcitabine and nab-paclitaxel, gemcitabine and cisplatin, gemcitabine and oxaliplatin, gemcitabine and capecitabine, and gemcitabine and oxaliplatin with or without nab-paclitaxel), oxaliplatin and/or nab-paclitaxel, 5-fluorouracil, 5-fluorouracil and oxaliplatin, or targeted cancer therapy, for example, EGF receptor targeted treatments such as erlotinib.

In some embodiments where ODSH is administered adjunctively with cancer treatment, ODSH is administered in combination with gemcitabine, optionally with nab-paclitaxel, to treat pancreatic cancer. The ODSH can be administered sequentially with gemcitabine and optional nab-paclitaxel. For example, ODSH can be administered as a bolus on the same day as, but before or after administration of gemcitabine and optional nab-paclitaxel. In one embodiment, ODSH is administered as a continuous infusion, starting either concurrently with the chemotherapeutic agent(s) or immediately thereafter, optionally preceded by a first “loading dose” of ODSH administered as a bolus. The bolus dose can be administered before or after administration of gemcitabine and optional nab-paclitaxel. Various regimens for administration of ODSH alone or in combination with chemotherapeutic agents are possible, some illustrations of which are further described in the Examples below. Additionally, regimens for chemotherapeutic agents used to treat pancreatic cancer, including gemcitabine or gemcitabine in combination with nab-paclitaxel have been described. See prescribing information for Gemzar®; Burris, H. A., et al., 1997, J. Clin. Oncol. 15(6):2403-13; and Von Hoff et al., 2011, J. Clinical Oncology, 29:1-8, for gemcitabine plus nab-paclitaxel regimen for the treatment of pancreatic cancer.

5.4. Resistance of Cancer to Treatment

ODSH has recently been demonstrated to inhibit binding of Receptor for Advanced Glycation End products (RAGE) by High Mobility Group Box 1 protein (HMGB1), a DNA-binding protein that functions as a cytokine, and other RAGE ligands. Rao, N. V., et al., 2010, Am. J. Physiol. Cell Physiol. 299:C97-C110, and WO 2009/015183, the contents of which are hereby incorporated by reference in their entirety. HMGB1 and RAGE, in turn, have been implicated in the regulation of autophagy which is associated with increased resistance of cancer cells to cytotoxic treatments. Without intending to be bound by theory, it is believed that the adjunctive administration of ODSH reduces the interaction of HMGB1 with RAGE, disrupting the cancer cell's ability to increase autophagy, thereby inhibiting de-sensitization or development of resistance, and maintaining sensitivity to the co-administered cytotoxic therapy. The therapeutically beneficial effects of adjunctively administered ODSH observed in the tumor xenograft model of pancreatic cancer are therefore of particular utility in the treatment of cancers that are, or can become, resistant to treatment via induction of autophagy.

5.5. Treatment of Breast Cancer

Breast cancer tumors can develop resistance to cancer treatment. At least one breast cancer cell line, when treated with an antibody against HER2, showed increased autophagy that correlated with resistance to HER2-targeted therapy. HMGB1 and RAGE are also expressed at higher than normal levels in breast cancer cell lines. As such, subjects suffering from breast cancer are candidates for treatment with ODSH. Accordingly, in another aspect, the methods of the present application comprise treating breast cancer by administering ODSH in combination with cancer treatment, as described above in Section 5.2.1, to a subject suffering from breast cancer. Alternatively, the methods of the present application comprise treating cancer by administering ODSH alone, as described above in Section 5.2.2, to a subject suffering from breast cancer.

ODSH can be administered to subjects with breast cancer to prevent resistance to cancer treatment or re-sensitize cancers that have acquired resistance to such treatments. In some embodiments, ODSH is administered in combination with chemotherapy. In some embodiments, ODSH is administered in combination with a targeted cancer therapy, such as trastuzumab or an anti-HER2 monoclonal antibody. In some embodiments, the ODSH is administered in combination with radiation therapy.

5.6. Treatment of Renal Cancer

In another aspect, the methods of the present application comprise treating renal cancer by administering ODSH in combination with cancer treatment, as described above in Section 5.2.1, to a subject suffering from renal cancer. Alternatively, the methods of the present application comprise treating cancer by administering ODSH alone, as described above in Section 5.2.2, to a subject suffering from renal cancer. ODSH can be administered to subjects with renal cancer to prevent resistance to cancer treatment or re-sensitize cancers that have acquired resistance to such treatments. In some embodiments, ODSH is administered in combination with chemotherapy. In some embodiments, ODSH is administered in combination with targeted cancer therapy, such as a tyrosine kinase inhibitor or an mTOR inhibitor. In some embodiments, ODSH is administered in combination with radiation therapy.

5.7. Treatment of Colorectal Cancer

In another aspect, the methods of the present application comprise treating colorectal cancer by administering ODSH in combination with cancer treatment, as described above in Section 5.2.1, to a subject suffering from colorectal cancer. Alternatively, the methods of the present application comprise treating cancer by administering ODSH alone, as described above in Section 5.2.2, to a subject suffering from colorectal cancer. ODSH can be administered to subjects with colorectal cancer to prevent resistance to cancer treatment or re-sensitize cancers that have acquired resistance to such treatments. In some embodiments, ODSH is administered in combination with a chemotherapeutic agent, including but not limited to 5-fluorouracil, 5-fluorouracil prodrugs, such as Xeloda®, irinotecan, leucovorin, and/or oxaliplatin, or any combination thereof, a targeted cancer therapy, including but not limited to the EGF receptor targeted therapy cetuximab and the VEGF targeted therapy bevacizumab, or radiation therapy.

5.8. Treatment of Leukemia

In another aspect, the methods of the present application comprise treating leukemia by administering ODSH in combination with cancer treatment, as described above in Section 5.2.1, to a subject suffering from leukemia. Alternatively, the methods of the present application comprise treating cancer by administering ODSH alone, as described above in Section 5.2.2, to a subject suffering from leukemia. ODSH can be administered to subjects with leukemia to prevent resistance to cancer treatment or re-sensitize cancers that have acquired resistance to such treatments. In some embodiments, ODSH is administered in combination with chemotherapeutic agents, including but not limited to cytosine arabinoside and daunorubicin or related anthracyclines, or any combination thereof, or targeted cancer therapy, including but not limited to tyrosine kinase targeted therapy, such as imatinib. In some embodiments, ODSH is administered in combination with radiation therapy.

5.9. Treatment of Gastric Cancer

In another aspect, the methods of the present application comprise treating gastric cancer by administering ODSH in combination with cancer treatment, as described above in Section 5.2.1, to a subject suffering from gastric cancer. Alternatively, the methods of the present application comprise treating cancer by administering ODSH alone, as described above in Section 5.2.2, to a subject suffering from gastric cancer. ODSH can be administered to subjects with gastric cancer to prevent resistance to cancer treatment or re-sensitize cancers that have acquired resistance to such treatments. In some embodiments, ODSH is administered in combination with chemotherapeutic agents, including but not limited to docetaxel, cisplatin, and/or 5-fluorouracil, or any combination thereof, or targeted cancer therapy, including but not limited to an anti-HER2 monoclonal antibody or an anti-VEGF monoclonal antibody. In some embodiments, ODSH is administered in combination with radiation therapy.

5.10. Treatment of Esophageal Cancer

In another aspect, the methods of the present application comprise treating esophageal cancer by administering ODSH alone or in combination with cancer treatment, as described above in Section 5.2.1, to a subject suffering from esophageal cancer. Alternatively, the methods of the present application comprise treating cancer by administering ODSH alone, as described above in Section 5.2.2, to a subject suffering from esophageal cancer. ODSH can be administered to subjects with esophageal cancer to prevent resistance to cancer treatment or re-sensitize cancers that have acquired resistance to such treatments. In some embodiments, ODSH is administered in combination with chemotherapeutic agents, including but not limited to docetaxel, cisplatin, and/or 5-fluorouracil, or any combination thereof. In some embodiments, ODSH is administered in combination with targeted cancer therapy, including but not limited to an anti-HER2 monoclonal antibody or an anti-VEGF monoclonal antibody. In some embodiments, ODSH is administered in combination with radiation therapy.

5.11. ODSH

ODSH for use in the above-described methods can be synthesized by cold alkaline hydrolysis of USP porcine intestinal heparin, which removes the 2-O and 3-O sulfates, leaving N- and 6-O sulfates and carboxylates substantially intact. Fryer, A. et al., 1997, J. Pharmacol. Exp. Ther. 282: 208-219. Using this method, ODSH can be produced with an average molecular mass of about 11.7±0.3 kg kDa, and low affinity for anti-thrombin III (Kd ˜339 μM or 4 mg/ml vs. 1.56 μM or 22 μg/ml for heparin), consistent with the observed low level of anticoagulant activity. Methods for the preparation of 2-O, 3-O desulfated non-anticoagulant heparin may also be found, for example, in U.S. Pat. Nos. 5,668,118, 5,912,237, and 6,489,311, and WO 2009/015183, the contents of which are incorporated in their entirety herein, and in U.S. Pat. Nos. 5,296,471, 5,969,100, and 5,808,021.

5.12. Pharmaceutical Compositions and Unit Dosage Forms

ODSH will generally be administered in the form of pharmaceutical formulations or compositions. Pharmaceutical compositions, suitable for administration to subjects, may optionally include additional active and/or therapeutic agents, as is known in the art. See Remington The Science and Practice of Pharmacy, 21^{st} Ed. (2005), Lippincott Williams & Wilkins, incorporated herein by reference. The formulations will typically include one or more pharmaceutically acceptable carriers, excipients, or diluents. The specific carriers, excipients, and/or diluents used will depend on the desired mode of administration.

Pharmaceutical compositions can be conveniently presented in unit dosage forms, which contain a predetermined amount of ODSH. Unit dosage forms can contain for example, but without limitation, 1 mg to 1 g, or 5 mg to 500 mg of ODSH.

The pharmaceutical compositions can be formulated for administration to subjects by a variety of routes, typically parenterally, including intravenous or subcutaneous administration. Pharmaceutical compositions can be formulated in volumes and concentrations suitable for bolus administration, for continuous infusion, or for subcutaneous administration. The pharmaceutical compositions may, for example, be in the form of a sterile, non-pyrogenic, fluid composition.

### 6. EXAMPLES

### Example 1

### In Vivo Evaluation of ODSH and Combined ODSH/chemotherapy treatment in the BxPC-3 Pancreatic Tumor Xenograft Model

This experiment demonstrates the effects of ODSH administered alone or in combination with chemotherapeutic agents on human pancreatic tumors growing as xenografts in athymic nude mice, including effects of ODSH on tumor growth inhibition.

### 1.1 Materials & Methods

Compounds tested in the experiment were as follows. ODSH was made by Pyramid Laboratories, Inc. (Costa Mesa, Calif.). ODSH was provided at a stock concentration of 50 mg/ml and stored at room temperature until use. ODSH was diluted in a 0.9% NaCl solution (B. Braun Medical Inc., Irvine, Calif.) to a concentration of 2.4 mg/ml to deliver 24 mg/kg, in a 10 ml/kg dose volume when administered intravenously. A concentration of 4.8 mg/ml was formulated to deliver a 24 mg/kg dose at a 5 ml/kg dose volume when administered subcutaneously. ODSH was formulated fresh prior to each dose. The chemotherapeutic agents oxaliplatin, gemcitabine, and nab-paclitaxel were also tested. Oxaliplatin was manufactured by Sanofi-Aventis (Bridgewater, N.J.) and diluted in a 0.9% NaCl solution to a concentration of 1 mg/ml to deliver 10 mg/kg, in a 10 ml/kg dose volume. Gemcitabine was manufactured by Eli Lilly and Co. (Indianapolis, Ind.) and diluted in a 0.9% NaCl solution to a concentration of 8 mg/ml to deliver 80 mg/kg, in a 10 ml/kg dose volume. Nab-paclitaxel was manufactured by Abraxis BioScience LLC (Bridgewater, N.J.) and diluted in a 0.9% NaCl solution to a concentration of 1.5 mg/ml to deliver 15 mg/kg, in a 10 ml/kg dose volume. All standard agent preparations were made fresh prior to their administration.

BxPC-3 cells were obtained and prepared as follows. The BxPC-3 pancreas tumor cell line was received from American Type Culture Collection (ATCC, Manassas, Va.). Cultures were maintained in RPMI 1640 medium (Hyclone, Logan, Utah) supplemented with 5% fetal bovine serum. The cells were housed in a 5% CO₂ atmosphere. The cultures were expanded in tissue culture flasks at a 1:3 split ratio until a sufficient amount of cells were harvested.

All experiments were conducted on female athymic nude mice (Hsd:Athymic Nude-Foxn1nu) supplied by Harlan (Indianapolis, Ind.). Mice were received at four weeks of age, 12-15 grams in weight, and were acclimated for seven days prior to handling. The mice were housed in microisolator cages (Lab Products, Seaford, Del.) and maintained under specific pathogen-free conditions. All procedures were carried out under appropriate institutional guidelines for animal care.

BxPC-3 Human Pancreas Tumor Xenograft Model: Female athymic nude mice per treatment condition were inoculated subcutaneously in the right flank with 0.1 ml of a 50% RPMI 1640/50% Matrigel™ (BD Biosciences, Bedford, Mass.) mixture containing a suspension of BxPC-3 tumor cells (approximately 5×10⁶ cells/mouse).

Seven days following inoculation, tumors were measured using calipers and tumor weight was calculated using the animal study management software, Study Director V.1.7.54 k (Study Log). See Britten C D, et al. Enhanced antitumor activity of 6-hydroxymethylacylfulvene in combination with irinotecan and 5-fluorouracil in the HT29 human colon tumor xenograft model. Cancer Res 59:1049-1053, 1999. Eighty mice with tumor sizes of 93-172 mg were placed into eight groups of ten mice by random equilibration (Day 1). Body weights were recorded when the mice were randomized and were taken twice weekly thereafter in conjunction with tumor measurements, on each of Days 1, 4, 8, 11, 15, 18, 22, 26, 30, 33, and 36.

ODSH, vehicle control (0.9% NaCl solution, referred to as saline), oxaliplatin, gemcitabine, and nab-paclitaxel were administered according to the dosing regimen described in Table 1. The study was terminated when the vehicle control reached an endpoint of 1500 mg, on Day 36. Table 1, below, provides further details on the eight treatment groups.

TABLE 1

| Treatment | | Agent | | |
|---|---|---|---|---|
| group | Treatment | administered | Dosing schedule and amount | Route* |
| 1 | Vehicle control | 0.9% saline | Twice a day, Day 1 to Day 11 | IV |
| | | | Twice a day, Day 12 to Day 35 | SC |
| 2 | ODSH | 24 mg/kg | Twice a day, Day 1 to Day 11 | IV |
| | | | Twice a day, Day 12 to Day 35 | SC |
| 3 | Oxaliplatin | 10 mg/kg | Once a week for 4 weeks (Days 1, 8, 15, 22) | IV |
| | Gemcitabine | 80 mg/kg | Every three days for 3 administrations (Days 26, 29, 32) | IP |
| | Nab-paclitaxel | 15 mg/kg | Every three days for 3 administrations (Days 26, 29, 32) | IV |
| 4 | Gemcitabine | 80 mg/kg | Every three days for 4 administrations (Days 1, 4, 7, 10) | IP |
| 5 | ODSH/ | 24 mg/kg | Twice a day, Day 1 to Day 11 | IV |
| | | | Twice a day, Day 12 to Day 35 | SC |
| | Oxaliplatin | 10 mg/kg | Once a week for 4 weeks (Days 1, 8, 15, 22) | IV |
| | Gemcitabine | 80 mg/kg | Every three days for 4 administrations (Days 26, 29, 32, 35) | IP |
| | Nab-paclitaxel | 15 mg/kg | Every three days for 3 administrations (Days 26, 29, 32) | IV |
| 6 | ODSH | 24 mg/kg | Twice a day, Day 1 to Day 11 | IV |
| | | | Twice a day, Day 12 to Day 35 | SC |
| | Gemcitabine | 80 mg/kg | Every three days for 4 administrations (Days 1, 4, 7, 10) | IP |
| 7 | Oxaliplatin | 10 mg/kg | Single administration (Day 1) | IV |
| | Gemcitabine | 80 mg/kg | Every three days for 3 administrations (Days 1, 4, 7) | IP |
| 8 | ODSH | 24 mg/kg | Twice a day, Day 1 to Day 8 | IV |
| | Oxaliplatin | 10 mg/kg | Single administration (Day 1) | IV |
| | Gemcitabine | 80 mg/kg | Every three days for 3 administrations (Days 1, 4, 7) | IP |

*Agents were administered by one or three routes: intravenous (IV), subcutaneous (SC), or intraperitoneal (IP).

Treatment for Groups 7 and 8 was ceased on Day 8 due to adverse effects resulting from the treatment. The ODSH dosing route was modified from intravenous to subcutaneous on Day 12, as a result of tail swelling and bruising. Gemcitabine and nab-paclitaxel were introduced into the dosing regimen of Groups 3 and 5 on Day 26.

Data and statistical analyses were performed as follows. Mean tumor growth inhibition (TGI) was calculated utilizing Formula A below (deaths were not included in the TGI calculations). TGI calculations were performed comparing tumor weights of Day 26 to Day 1, which captures data prior to the addition of gemcitabine and nab-paclitaxel to several groups, and Day 36 (final day of study) to Day 1.

$$TGI = [ 1 - \frac{( {\overline{χ}}_{{Treated}_{( Final )}} - {\overline{χ}}_{{Treated}_{( Day 1 )}} )}{( {\overline{χ}}_{{Control}_{( Final )}} - {\overline{χ}}_{{Control}_{( Day 1 )}} )} ] \times 100 \% \qquad Formula A$$

All statistical analyses in the xenograft study were performed with GraphPad Prism® v4 software. Differences in Day 26 and 36 tumor weights were confirmed using the Analysis of Variance (ANOVA) with the Tukey's Multiple Comparison Test.

### 1.2 Results

The antitumor effects of ODSH administered as a single agent or in various combinations with one or more of oxaliplatin, gemcitabine, and nab-paclitaxel were evaluated.

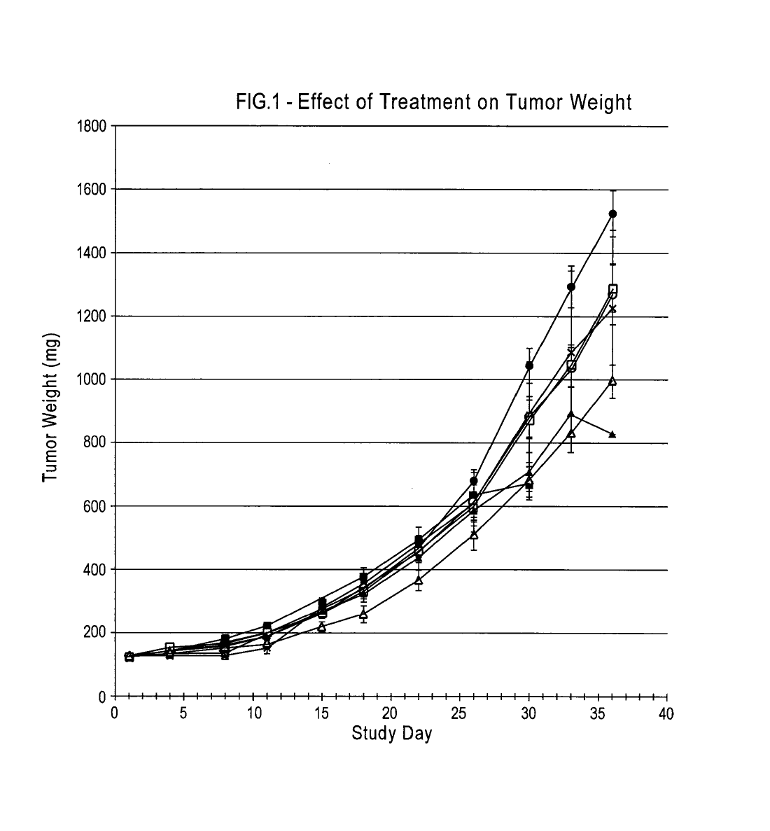

The recorded tumor weights for experimental treatment groups 1 through 8 are provided below in Tables 2 through 9. See also FIG. 1.

TABLE 2
Group 1

| | | | | | | Dose Route*: Intravenous/Subcutaneous | | | | | |
|---|---|---|---|---|---|---|---|---|---|---|---|
| | PBS Control (0 mg/kg) | | | | | Frequency: BID to end | | | | | |
| | Day: | | | | | | | | | | |
| | 1 | 4 | 8 | 11 | 15 | 18 | 22 | 26 | 30 | 33 | 36 |
| Mouse 1 | 106 | 127 | 155 | 182 | 293 | 365 | 484 | 766 | 4161 | 4627 | 1,707 |
| Mouse 2 | 114 | 140 | 195 | 190 | 267 | 321 | 426 | 609 | 836 | 1,060 | 1379.0295 |
| Mouse 3 | 106 | 119 | 166 | 194 | 223 | 300 | 420 | 635 | 1,067 | 1,306 | 1689.984 |
| Mouse 4 | 93 | 103 | 120 | 160 | 237 | 338 | 449 | 681 | 1,132 | 1,311 | 1565.568 |
| Mouse 5 | 140 | 145 | 207 | 249 | 306 | 346 | 546 | 780 | 1,020 | 1,230 | 13814605 |
| Mouse 6 | 114 | 118 | 138 | 154 | 218 | 273 | 344 | 557 | 722 | 986 | 1295.6555 |
| Mouse 7 | 129 | 159 | 200 | 194 | 282 | 321 | 461 | 681 | 1,054 | 1,286 | 1,352 |
| Mouse 8 | 130 | 122 | 134 | 176 | 272 | 320 | 415 | 583 | 999 | 1,165 | 1,329 |
| Mouse 9 | 142 | 153 | 164 | 184 | 318 | 333 | 465 | 590 | 1,080 | 1,341 | 1,522 |
| Mouse 10 | 172 | 194 | 259 | 285 | 377 | 505 | 665 | 915 | 1,362 | 1,620 | 2,020 |
| Mean | 124.6 | 138.1 | 173.7 | 196.9 | 279.2 | 342.2 | 467.6 | 679.8 | 1,043.2 | 1,293.3 | 1,524.2 |
| Median | 121.6 | 133.7 | 164.8 | 186.9 | 276.8 | 327.3 | 454.9 | 657.9 | 1,060.4 | 1,296.2 | 1,451.9 |
| Std Dev | 22.78 | 26.24 | 41.72 | 40.36 | 48.10 | 62.40 | 86.85 | 111.90 | 174.53 | 208.14 | 228.53 |
| Std Err | 7.20 | 8.30 | 13.19 | 12.76 | 15.21 | 19.73 | 27.46 | 35.39 | 55.19 | 65.82 | 72.27 |

*ODSH dosed intravenously Days 1-11; dosed subcutaneously Days 12-end

TABLE 3
Group 2

| | | | | | | Dose Route*: Intravenous/Subcutaneous | | | | | |
|---|---|---|---|---|---|---|---|---|---|---|---|
| | ODSH (24 mg/kg) | | | | | Frequency: BID to end | | | | | |
| | Day: | | | | | | | | | | |
| | 1 | 4 | 8 | 11 | 15 | 18 | 22 | 26 | 30 | 33 | 36 |
| Mouse 1 | 143 | 144 | 157 | 140 | 218 | 278 | 326 | 394 | 555 | 733 | 836 |
| Mouse 2 | 129 | 133 | 148 | 203 | 264 | 352 | 515 | 766 | 1,109 | 1,340 | 1,823 |
| Mouse 3 | 140 | 162 | 218 | 149 | 206 | 278 | 358 | 509 | 697 | 908 | 1,072 |
| Mouse 4 | 114 | 98 | 118 | 288 | 340 | 489 | 622 | 894 | 1,083 | 1,218 | 1,450 |
| Mouse 5 | 116 | 110 | 144 | 142 | 224 | 291 | 360 | 478 | 756 | 999 | 1,153 |
| Mouse 6 | 172 | 141 | 183 | 175 | 263 | 288 | 400 | 529 | 893 | 961 | 1,150 |
| Mouse 7 | 94 | 97 | 125 | 243 | 348 | 419 | 556 | 770 | 1,201 | 1,309 | 1,654 |
| Mouse 8 | 131 | 181 | 222 | 146 | 237 | 256 | 402 | 573 | 820 | 961 | 1,150 |
| Mouse 9 | 106 | 135 | 126 | 252 | 321 | 415 | 551 | 724 | 945 | 1,087 | 1,376 |
| Mouse 10 | 125 | 130 | 155 | 137 | 208 | 318 | 449 | 524 | 764 | 871 | 1,037 |
| Mean | 122.4 | 134.0 | 146.0 | 187.6 | 263.0 | 338.5 | 453.8 | 616.2 | 882.3 | 1,038.6 | 1,270.1 |
| Median | 22.9 | 28.6 | 40.4 | 162.1 | 250.0 | 304.7 | 425.4 | 551.3 | 856.4 | 979.8 | 1,151.5 |
| Std Dev | 7.24 | 9.03 | 12.76 | 55.55 | 54.66 | 77.75 | 101.08 | 160.86 | 203.52 | 197.30 | 302.01 |
| Std Err | | | | 17.57 | 17.29 | 24.59 | 31.96 | 50.87 | 64.36 | 62.39 | 95.50 |

*ODSH dosed intravenously Days 1-11; dosed subcutaneously Days 12-end

TABLE 4
Group 3\hat{ }

| | | | | | | Dose Route: Intravenous | | | | | |
|---|---|---|---|---|---|---|---|---|---|---|---|
| | Oxaliplatin 10 mg/kg | | | | | Frequency: Wkly × 4 (Day 1, 8, 15, 22) | | | | | |
| | | | | | | Dose Route: Intraperitoneal | | | | | |
| | | | | | | Frequency: Day 26, 39, 32 | | | | | |
| | Gemcitabine 80 mg/kg | | | | | (Q3d×0 3 starting Day 26) | | | | | |
| | | | | | | Dose Route: Intravenous | | | | | |
| | | | | | | Frequency: Day 26, 29, 32 | | | | | |
| | Nab/paclitaxel 15 mg/kg | | | | | (2 × weekly starting Day 26) | | | | | |
| | Day: | | | | | | | | | | |
| | 1 | 4 | 8 | 11 | 15 | 18 | 22 | 26 | 30 | 33 | 36 |
| Mouse 1 | 113 | 144 | 154 | 202 | 271 | 300 | 392 | 570 | 725 | FD | FD |
| Mouse 2 | 131 | 135 | 219 | 184 | 250 | 269 | 322 | 397 | 393 | MS | MS |
| Mouse 3 | 104 | 113 | 142 | 164 | 219 | 289 | 379 | 487 | 595 | FD | FD |
| Mouse 4 | 143 | 148 | 205 | 265 | 368 | 485 | 611 | 812 | FD | FD | FD |
| Mouse 5 | 168 | 169 | 245 | 299 | 394 | 533 | 673 | 918 | FD | FD | FD |
| Mouse 6 | 128 | 114 | 167 | 201 | 282 | 314 | 451 | 522 | 658 | FD | FD |
| Mouse 7 | 96 | 126 | 161 | 211 | 263 | 362 | 432 | 629 | 687 | FD | FD |
| Mouse 8 | 116 | 136 | 205 | 243 | 308 | 461 | 666 | 824 | 926 | FD | FD |
| Mouse 9 | 106 | 139 | 165 | 222 | 301 | 371 | 522 | 600 | 706 | MS | MS |
| Mouse 10 | 139 | 133 | 157 | 217 | 282 | 394 | 495 | 589 | 689 | MS | MS |
| Mean | 124.5 | 135.7 | 182.0 | 220.6 | 293.7 | 377.6 | 494.4 | 635.0 | 672.2 | | |
| Median | 121.9 | 135.7 | 165.8 | 213.6 | 282.0 | 366.4 | 473.1 | 594.5 | 688.0 | | |
| Std Dev | 21.78 | 16.25 | 33.99 | 39.41 | 52.76 | 90.07 | 122.52 | 165.28 | 147.79 | | |
| Std Err | 6.89 | 5.14 | 10.75 | 12.46 | 16.68 | 28.48 | 38.74 | 52.26 | 52.25 | | |

\hat{ }Beginning Day 26, groups 3 and 5 were taken off initial dosing regimen and the following dosing regimen was initiated:
Gr 3: Gemcitabine (80 mg/kg IP, Q3d × 4 starting Day 26) + Nab-paclitaxel (15 mg/kg IV, 2 × weekly starting Day 26)
Gr 5: ODSH (24 mg/kg, IV BID) + Gemcitabine (80 mg/kg IP, Q3d × 4 starting Day 26) + Nab-paclitaxel (15 mg/kg IV, 2 × weekly starting day 26)

TABLE 5
Group 4

| | | | | | | Dose Route: Intraperitoneal | | | | | |
|---|---|---|---|---|---|---|---|---|---|---|---|
| | Gemcitabine 80 mg/kg | | | | | Frequency: Q3d × 4 (Day 1, 4, 7, 10) | | | | | |
| | Day: | | | | | | | | | | |
| | 1 | 4 | 8 | 11 | 15 | 18 | 22 | 26 | 30 | 33 | 36 |
| Mouse 1 | 146 | 155 | 180 | 234 | 300 | 389 | 500 | 647 | 992 | 1,158 | 1,267 |
| Mouse 2 | 96 | 135 | 134 | 180 | 274 | 304 | 404 | 583 | 826 | 1,008 | 1,265 |
| Mouse 3 | 132 | 189 | 193 | 235 | 319 | 413 | 621 | 797 | 1,121 | 1,367 | 1,573 |
| Mouse 4 | 139 | 214 | 282 | 328 | 375 | 379 | 514 | 739 | 1,008 | 1,132 | 1,368 |
| Mouse 5 | 126 | 126 | 129 | 146 | 211 | 258 | 372 | 468 | 674 | 878 | 963 |
| Mouse 6 | 104 | 122 | 114 | 145 | 218 | 267 | 400 | 534 | 860 | 1,036 | 1,347 |
| Mouse 7 | 107 | 131 | 130 | 119 | 181 | 224 | 350 | 431 | 667 | 936 | 1,106 |
| Mouse 8 | 111 | 133 | 125 | 150 | 228 | 269 | 332 | 389 | 589 | 624 | 923 |
| Mouse 9 | 116 | 146 | 161 | 202 | 245 | 441 | 592 | 730 | 1,150 | 1,275 | 1,596 |
| Mouse 10 | 166 | 176 | 175 | 242 | 288 | 376 | 509 | 628 | 856 | 1,030 | 1,482 |
| Mean | 124.3 | 152.5 | 162.2 | 198.2 | 263.8 | 332.1 | 459.4 | 594.7 | 874.3 | 1,044.2 | 1,288.8 |
| Median | 121.1 | 140.2 | 147.4 | 191.0 | 259.2 | 340.1 | 451.9 | 605.8 | 858.0 | 1,033.3 | 1,306.7 |
| Std Dev | 21.73 | 30.57 | 50.04 | 63.22 | 58.46 | 76.00 | 101.69 | 138.55 | 193.10 | 209.66 | 234.90 |
| Std Err | 6.87 | 9.67 | 15.83 | 19.99 | 18.49 | 24.03 | 32.16 | 43.81 | 61.06 | 66.30 | 74.28 |

TABLE 6
Group 5\hat{ }

| | | | | | | Dose Route*: Intravenous/Subcutaneous | | | | | |
|---|---|---|---|---|---|---|---|---|---|---|---|
| | ODSH 24 mg/kg | | | | | Frequency: BID to end | | | | | |
| | | | | | | Dose Route: Intravenous | | | | | |
| | Oxaliplatin 10 mg/kg | | | | | Frequency: Wkly × 4 (Day 1, 8, 15, 22) | | | | | |
| | | | | | | Dose Route: Intraperitoneal | | | | | |
| | | | | | | Frequency: Day 26, 29, 32, 35 | | | | | |
| | Gemcitabine 80 mg/kg | | | | | (Q3d × 4 starting Day 26) | | | | | |
| | | | | | | Dose Route: Intravenous | | | | | |
| | | | | | | Frequency: Day 26, 29, 32 | | | | | |
| | Nab/paclitaxel 15 mg/kg | | | | | (2 × weekly starting Day 26) | | | | | |
| | Day: | | | | | | | | | | |
| | 1 | 4 | 8 | 11 | 15 | 18 | 22 | 26 | 30 | 33 | 36 |
| Mouse 1 | 139 | 144 | 207 | 237 | 395 | 448 | 571 | 819 | 908 | FD | FD |
| Mouse 2 | 103 | 106 | 172 | 230 | 298 | 372 | 403 | 609 | 735 | FD | FD |
| Mouse 3 | 160 | 162 | 166 | 205 | 279 | 337 | 484 | 635 | 758 | FD | FD |
| Mouse 4 | 97 | 113 | 108 | 113 | 160 | 173 | 225 | 313 | 359 | FD | FD |
| Mouse 5 | 125 | 134 | 174 | 163 | 245 | 299 | 421 | 576 | 772 | FD | FD |
| Mouse 6 | 117 | 137 | 124 | 165 | 229 | 259 | 334 | 416 | 552 | MS | MS |
| Mouse 7 | 148 | 191 | 186 | 184 | 286 | 337 | 470 | 642 | 745 | 891 | 828 |
| Mouse 8 | 108 | 136 | 141 | 198 | 280 | 386 | 539 | 766 | MS | MS | MS |
| Mouse 9 | 133 | 177 | 214 | 226 | 313 | 400 | 578 | 631 | 834 | FD | FD |
| Mouse 10 | 111 | 119 | 156 | 173 | 219 | 229 | 333 | 457 | FD | FD | FD |
| Mean | 124.1 | 142.0 | 164.9 | 189.5 | 270.4 | 324.0 | 436.0 | 586.6 | 708.0 | 891.1 | 827.5 |
| Median | 121.2 | 136.4 | 169.2 | 191.2 | 279.4 | 336.7 | 445.8 | 620.2 | 751.6 | 891.1 | 877.5 |
| Std Dev | 20.52 | 27.53 | 34.05 | 37.97 | 62.97 | 84.34 | 115.23 | 154.52 | 173.64 | | |
| Std Err | 6.49 | 8.71 | 10.77 | 12.01 | 19.91 | 26.67 | 36.44 | 48.86 | 61.39 | | |

\hat{ }Beginning Day 26, groups 3 and 5 were taken off initial dosing regimen and the following dosing regimen was initiated:
Gr 3: Gemcitabine (80 mg/kg IP, Q3d × 4 starting Day 26) + Nab-paclitaxel (15 mg/kg IV, 2 × weekly starting Day 26)
Gr 5: ODSH (24 mg/kg, IV BID) + Gemcitabine (80 mg/kg IP, Q3d × 4 starting Day 26) + Nab-paclitaxel (15 mg/kg IV, 2 × weekly starting day 26)
*ODSH dosed intravenously Days 1-11; dosed subcutaneously Days 12-end
MS = Moribund Sacrifice
FD = Found Dead

TABLE 7
Group 6

| | | | | | | Dose Route*: Intravenous/Subcutaneous | | | | | |
|---|---|---|---|---|---|---|---|---|---|---|---|
| | ODSH 24 mg/kg | | | | | Frequency: BID to end | | | | | |
| | | | | | | Dose Route: Intraperitoneal | | | | | |
| | Gemcitabine 80 mg/kg | | | | | Frequency: Q3d × 4 (Day 1, 4, 7, 10) | | | | | |
| | Day: | | | | | | | | | | |
| | 1 | 4 | 8 | 11 | 15 | 18 | 22 | 26 | 30 | 33 | 36 |
| Mouse 1 | 160 | 169 | 194 | 135 | 203 | 279 | 407 | 562 | 780 | 893 | 1,115 |
| Mouse 2 | 101 | 92 | 113 | 151 | 188 | 194 | 309 | 374 | 497 | 578 | 862 |
| Mouse 3 | 119 | 160 | 170 | 209 | 278 | 257 | 326 | 452 | 571 | 822 | 997 |
| Mouse 4 | 134 | 122 | 128 | 154 | 188 | 231 | 330 | 409 | 595 | 773 | 834 |
| Mouse 5 | 125 | 134 | 141 | 152 | 197 | 265 | 332 | 520 | 622 | 856 | 965 |
| Mouse 6 | 97 | 102 | 117 | 156 | 203 | 229 | 336 | 491 | 746 | 820 | 1,048 |
| Mouse 7 | 137 | 121 | 166 | 171 | 205 | 256 | 389 | 510 | 704 | 789 | 868 |
| Mouse 8 | 151 | 174 | 203 | 255 | 331 | 484 | 635 | 886 | 1,069 | 1,289 | 1,365 |
| Mouse 9 | 111 | 120 | 123 | 130 | 149 | 199 | 273 | 342 | 490 | 631 | 833 |
| Mouse 10 | 108 | 134 | 131 | 118 | 239 | 191 | 323 | 543 | 757 | 843 | 1,051 |
| Mean | 124.2 | 132.7 | 148.7 | 163.0 | 218.1 | 258.5 | 336.0 | 508.9 | 683.3 | 829.3 | 993.9 |
| Median | 121.7 | 128.1 | 136.1 | 153.3 | 203.0 | 243.2 | 331.0 | 500.3 | 663.0 | 820.9 | 981.0 |
| Std Dev | 20.92 | 27.40 | 32.50 | 40.78 | 52.12 | 85.24 | 101.65 | 151.31 | 171.22 | 189.79 | 164.57 |
| Std Err | 6.61 | 8.67 | 10.28 | 12.90 | 16.48 | 26.96 | 32.15 | 47.85 | 54.15 | 60.02 | 52.04 |

*ODSH dosed intravenously Days 1-11; dosed subcutaneously Days 12-end

TABLE 8
Group 7

| | | | | | | Dose Route: Intravenous | | | | | |
|---|---|---|---|---|---|---|---|---|---|---|---|
| | Oxaliplatin 10 mg/kg | | | | | Frequency: Day 1 | | | | | |
| | | | | | | Dose Route: Intraperitoneal | | | | | |
| | Gemcitabine 80 mg/kg | | | | | Frequency: Q3d × 3 (Day 1, 4, 7) | | | | | |
| | Day: | | | | | | | | | | |
| | 1 | 4 | 8 | 11 | 15 | 18 | 22 | 26 | 30 | 33 | 36 |
| Mouse 1 | 137 | 119 | 132 | 110 | MS | MS | MS | MS | MS | MS | MS |
| Mouse 2 | 159 | 175 | 165 | 197 | 259 | 368 | 453 | 527 | 735 | 787 | 877 |
| Mouse 3 | 110 | 104 | 107 | 137 | MS | MS | MS | MS | MS | MS | MS |
| Mouse 4 | 110 | 114 | 110 | 117 | MS | MS | MS | MS | MS | MS | MS |
| Mouse 5 | 135 | 117 | 81 | FD | FD | FD | FD | FD | FD | FD | FD |
| Mouse 6 | 97 | 98 | 103 | 106 | FD | FD | FD | FD | FD | FD | FD |
| Mouse 7 | 152 | 173 | 222 | 217 | 314 | 389 | 541 | 801 | 1,221 | 1,599 | 1,699 |
| Mouse 8 | 121 | 117 | 113 | FD | FD | FD | FD | FD | FD | FD | FD |
| Mouse 9 | 119 | 146 | 150 | 173 | 272 | 299 | 424 | 510 | 715 | 871 | 1,107 |
| Mouse 10 | 100 | 111 | 103 | FD | FD | FD | FD | FD | FD | FD | FD |
| Mean | 124.1 | 127.2 | 128.7 | 150.9 | 281.7 | 352.1 | 472.7 | 612.4 | 890.1 | 1,085.5 | 1,227.7 |
| Median | 120.1 | 116.6 | 111.8 | 136.8 | 272.0 | 368.5 | 452.8 | 526.9 | 734.8 | 870.9 | 1,107.0 |
| Std Dev | 21.16 | 27.63 | 41.13 | 44.59 | 29.01 | 47.57 | 60.91 | 163.25 | 286.32 | 446.77 | 423.84 |
| Std Err | 6.69 | 8.74 | 13.01 | 16.85 | 16.75 | 27.46 | 36.17 | 94.25 | 165.31 | 257.94 | 244.71 |

MS = Moribund Sacrifice
FD = Found Dead

TABLE 9
Group 8

| | | | | | | Dose Route*: Intravenous/Subcutaneous | | | | | |
|---|---|---|---|---|---|---|---|---|---|---|---|
| | ODSH 24 mg/kg | | | | | Frequency: BID × 8 days | | | | | |
| | | | | | | Dose Route: Intravenous | | | | | |
| | Oxaliplatin 10 mg/kg | | | | | Frequency: Day 1 | | | | | |
| | | | | | | Dose Route: Intraperitoneal | | | | | |
| | Gemcitabine 80 mg/kg | | | | | Frequency: Q3d × 3 (Day 1, 4, 7) | | | | | |
| | Day: | | | | | | | | | | |
| | 1 | 4 | 8 | 11 | 15 | 18 | 22 | 26 | 30 | 33 | 36 |
| Mouse 1 | 121 | 129 | 127 | FD | FD | FD | FD | FD | FD | FD | FD |
| Mouse 2 | 153 | 147 | 94 | FD | FD | FD | FD | FD | FD | FD | FD |
| Mouse 3 | 157 | 174 | 183 | FD | FD | FD | FD | FD | FD | FD | FD |
| Mouse 4 | 110 | 102 | 81 | FD | FD | FD | FD | FD | FD | FD | FD |
| Mouse 5 | 121 | 123 | FD | FD | FD | FD | FD | FD | FD | FD | FD |
| Mouse 6 | 110 | 124 | 127 | FD | FD | FD | FD | FD | FD | FD | FD |
| Mouse 7 | 99 | 82 | 121 | FD | FD | FD | FD | FD | FD | FD | FD |
| Mouse 8 | 135 | 152 | 148 | FD | FD | FD | FD | FD | FD | FD | FD |
| Mouse 9 | 136 | 159 | 215 | 196 | MS | MS | MS | MS | MS | MS | MS |
| Mouse 10 | 99 | 118 | 122 | FD | FD | FD | FD | FD | FD | FD | FD |
| Mean | 124.1 | 131.0 | 135.4 | 196.0 | | | | | | | |
| Median | 121.0 | 126.3 | 127.0 | 196.0 | | | | | | | |
| Std Dev | 20.79 | 27.70 | 4.84 | | | | | | | | |
| Std Err | 6.57 | 8.76 | 13.95 | | | | | | | | |

FD = Found Dead
MS = Moribund Sacrifice
*ODSH dosed intravenously Days 1-11; dosed subcutaneously Days 12-end
\hat{ }Beginning Day 26, groups 3 and 5 were taken off initial dosing regimen and the following dosing regimen was initiated:
Gr 3: Gemcitabine (80 mg/kg IP, Q3d × 4 starting Day 26) + Nab-paclitaxel (15 mg/kg IV, 2 × weekly starting Day 26)
Gr 5: ODSH (24 mg/kg, IV BID) + Gemcitabine (80 mg/kg IP, Q3d × 4 starting Day 26) + Nab-paclitaxel (15 mg/kg IV, 2 × weekly starting day 26)

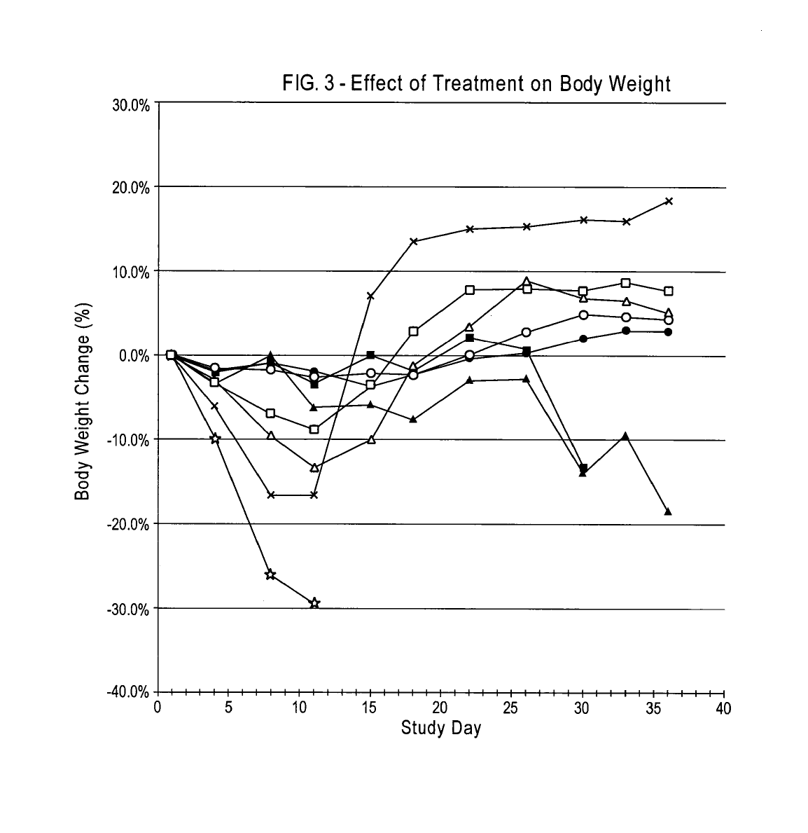

Tables 10 to 13 below show the body weights recorded for treatment groups 1 to 8 over the course of the experiment. See also FIG. 3.

TABLE 10

| | | | | | | | | Day 4 | | | Day 8 | | |
|---|---|---|---|---|---|---|---|---|---|---|---|---|---|
| | | | | | Day 1 | | | | | % | | | % |
| | | | | | Starting | | Average | | Average | Weight | | Average | Weight |
| | | | | | Weight | # | Weight | # | Weight | Loss or | # | Weight | Loss or |
| Group | Compound | Dosage | Frequency | Dose Route* | (g) | Mice | (g) | Mice | (g) | Gain | Mice | (g) | Gain |
| 1 | Vehicle | 0 mg/kg | BID to end | Intravenous/ | 20.86 | 10 | 20.86 | 10 | 20.48 | −1.82 | 10 | 20.67 | −0.91 |
| | Control | | | Subcutaneous | | | | | | | | | |
| 2 | ODSH | 24 mg/kg | BID to end | Intravenous/ | 21.54 | 10 | 21.54 | 10 | 21.18 | −1.67 | 10 | 21.18 | −1.67 |
| | | | | Subcutaneous | | | | | | | | | |
| 3\hat{ } | Oxaliplatin | 10 mg/kg | Wkly × 4 (Day | Intravenous | 20.82 | 10 | 20.82 | 10 | 20.40 | −2.02 | 10 | 20.65 | −0.82 |
| | | | 1, 8, 15, 22) | | | | | | | | | | |
| 4 | Gemcitabine | 80 mg/kg | Q3d × 4 (Day | Intraperitoneal | 22.48 | 10 | 22.48 | 10 | 21.76 | −3.20 | 10 | 20.91 | −6.98 |
| | | | 1, 4, 7, 10) | | | | | | | | | | |
| 5\hat{ } | ODSH + | 24 mg/kg + | BID to end + | Intravenous/ | 21.11 | 10 | 21.11 | 10 | 20.40 | −3.36 | 10 | 21.08 | −0.14 |
| | Oxaliplatin | 10 mg/kg | Wkly × 4 (Day | Subcutaneous | | | | | | | | | |
| | | | 1, 8, 15, 22) | | | | | | | | | | |
| 6 | ODSH + | 24 mg/kg + | BID to end + | Intravenous/ | 20.95 | 10 | 20.95 | 10 | 20.31 | −3.05 | 10 | 18.92 | −9.69 |
| | Gemcitabine | 80 mg/kg | Q3d × 4 (Day | Subcutaneous + | | | | | | | | | |
| | | | 1, 4, 7, 10) | Intravenous | | | | | | | | | |
| 7 | Oxaliplatin + | 10 mg/kg + | Day 1 + Q3d × | Intravenous + | 20.58 | 10 | 20.58 | 10 | 19.32 | −6.12 | 10 | 17.17 | −16.57 |
| | Gemcitabine | 80 mg/kg | 3 (Day 1, 4, 7) | Intravenous | | | | | | | | | |
| 8 | ODSH + | 24 mg/kg + | BID × 8 days + | Intravenous + | 20.96 | 10 | 20.96 | 10 | 18.88 | −9.92 | 9 | 15.50 | −26.05 |
| | Oxaliplatin + | 10 mg/kg + | Day 1 + Q3d × | Intravenous + | | | | | | | | | |
| | Gemcitabine | 80 mg/kg | 3 (Day 1, 4, 7) | Intraperitoneal | | | | | | | | | |

\hat{ }Beginning Day 26, groups 3 and 5 were taken off initial dosing regimen and the following dosing regimen was initiated:
Gr 3: Gemcitabine (80 mg/kg IP, Q3d × 4 starting Day 26) + Nab-paclitaxel (15 mg/kg IV, 2 × weekly starting Day 26)
Gr 5: ODSH (24 mg/kg, IV BID) + Gemcitabine (80 mg/kg IP, Q3d × 4 starting Day 26) + Nab-paclitaxel (15 mg/kg IV, 2 × weekly starting day 26)
*ODSH dosed intravenously Days 1-11; dosed subcutaneously Days 12-end

TABLE 11

| | | | | | Day 11 | | | Day 15 | | | Day 18 | | |
|---|---|---|---|---|---|---|---|---|---|---|---|---|---|
| | | | | | | | % | | | % | | | % |
| | | | | | | Average | Weight | | Average | Weight | | Average | Weight |
| | | | | | # | Weight | Loss or | # | Weight | Loss or | # | Weight | Loss or |
| Group | Compound | Dosage | Frequency* | Dose Route | Mice | (g) | Gain | Mice | (g) | Gain | Mice | (g) | Gain |
| 1 | Vehicle | 0 mg/kg | BID to end | Intravenous/ | 10 | 20.45 | −1.97 | 10 | 20.10 | −3.64 | 10 | 20.36 | −2.40 |
| | Control | | | Subcutaneous | | | | | | | | | |
| 2 | ODSH | 24 mg/kg | BID to end | Intravenous/ | 10 | 20.95 | −2.74 | 10 | 21.08 | −2.14 | 10 | 21.04 | −2.32 |
| | | | | Subcutaneous | | | | | | | | | |
| 3\hat{ } | Oxaliplatin | 10 mg/kg | Wkly × 4 (Day | Intravenous | 10 | 20.12 | −3.36 | 10 | 20.81 | −0.05 | 10 | 20.45 | −1.78 |
| | | | 1, 8, 15, 22) | | | | | | | | | | |
| 4 | Gemcitabine | 80 mg/kg | Q3d × 4 (Day | Intraperitoneal | 10 | 20.52 | −8.72 | 10 | 21.70 | −3.47 | 10 | 23.15 | 2.98 |
| | | | 1, 4, 7, 10) | | | | | | | | | | |
| 5\hat{ } | ODSH + | 24 mg/kg + | BID to end + | Intravenous/ | 10 | 19.79 | −6.25 | 10 | 19.87 | −5.87 | 10 | 19.49 | −7.67 |
| | Oxaliplatin | 10 mg/kg | Wkly × 4 (Day | Subcutaneous + | | | | | | | | | |
| | | | 1, 8, 15, 22) | Intravenous | | | | | | | | | |
| 6 | ODSH + | 24 mg/kg + | BID to end + | Intravenous/ | 10 | 18.14 | −13.41 | 10 | 18.84 | −10.07 | 10 | 20.68 | −1.29 |
| | Gemcitabine | 80 mg/kg | Q3d × 4 (Day | Subcutaneous + | | | | | | | | | |
| | | | 1, 4, 7, 10) | Intravenous | | | | | | | | | |
| 7 | Oxaliplatin + | 10 mg/kg + | Day 1 + Q3d × | Intravenous + | 7 | 17.17 | −16.56 | 3 | 22.03 | 7.06 | 3 | 23.37 | 13.54 |
| | Gemcitabine | 80 mg/kg | 3 (Day 1, 4, 7) | Intravenous | | | | | | | | | |
| 8 | ODSH + | 24 mg/kg + | BID × 8 days + | Intravenous + | 1 | 14.80 | −29.39 | 0 | | | 0 | | |
| | Oxaliplatin + | 10 mg/kg + | Day 1 + Q3d × | Intravenous + | | | | | | | | | |
| | Gemcitabine | 80 mg/kg | 3 (Day 1, 4, 7) | Intraperitoneal | | | | | | | | | |

\hat{ }Beginning Day 26, groups 3 and 5 were taken off initial dosing regimen and the following dosing regimen was initiated:
Gr 3: Gemcitabine (80 mg/kg IP, Q3d × 4 starting Day 26) + Nab-paclitaxel (15 mg/kg IV, 2 × weekly starting Day 26)
Gr 5: ODSH (24 mg/kg, IV BID) + Gemcitabine (80 mg/kg IP, Q3d × 4 starting Day 26) + Nab-paclitaxel (15 mg/kg IV, 2 × weekly starting day 26)
*ODSH dosed intravenously Days 1-11; dosed subcutaneously Days 12-end

TABLE 12

| | | | | | Day 22 | | | Day 26 | | | Day 30 | | |
|---|---|---|---|---|---|---|---|---|---|---|---|---|---|
| | | | | | | | % | | | % | | | % |
| | | | | | | Average | Weight | | Average | Weight | | Average | Weight |
| | | | | | # | Weight | Loss or | # | Weight | Loss or | # | Weight | Loss or |
| Group | Compound | Dosage | Frequency* | Dose Route | Mice | (g) | Gain | Mice | (g) | Gain | Mice | (g) | Gain |
| 1 | Vehicle | 0 mg/kg | BID to end | Intravenous/ | 10 | 20.81 | −0.24 | 10 | 20.93 | 0.34 | 10 | 21.28 | 2.01 |
| | Control | | | Subcutaneous | | | | | | | | | |
| 2 | ODSH | 24 mg/kg | BID to end | Intravenous/ | 10 | 21.56 | 0.09 | 10 | 22.14 | 2.79 | 10 | 22.60 | 4.92 |
| | | | | Subcutaneous | | | | | | | | | |
| 3\hat{ } | Oxaliplatin | 10 mg/kg | Wkly × 4 (Day | Intravenous | 10 | 21.27 | 2.16 | 10 | 20.96 | 0.67 | 8 | 18.08 | −13.18 |
| | Gemcitabine | 80 mg/kg | 1, 8, 15, 22) | Intraperitoneal | | | | | | | | | |
| | Nab- | 15 mg/kg | Day 26, 29, 32 | Intravenous | | | | | | | | | |
| | paclitaxel | | Day 26, 29, 32 | | | | | | | | | | |
| 4 | Gemcitabine | 80 mg/kg | Q3d × 4 (Day | Intraperitoneal | 10 | 24.22 | 7.74 | 10 | 24.27 | 7.96 | 10 | 24.19 | 7.61 |
| | | | 1, 4, 7, 10) | | | | | | | | | | |
| 5\hat{ } | ODSH + | 24 mg/kg + | BID to end + | Intravenous/ | 10 | 20.47 | −3.03 | 10 | 20.51 | −2.84 | 8 | 18.16 | −13.96 |
| | Oxaliplatin | 10 mg/kg | Wkly × 4 (Day | Subcutaneous + | | | | | | | | | |
| | Gemcitabine | 80 mg/kg | 1, 8, 15, 22) | Intravenous | | | | | | | | | |
| | Nab- | 15 mg/kg | Day 26, 29, | Intraperitoneal | | | | | | | | | |
| | paclitaxel | | 32, 35 Day 26, | Intravenous | | | | | | | | | |
| | | | 29, 32 | | | | | | | | | | |
| 6 | ODSH + | 24 mg/kg + | BID to end + | Intravenous/ | 10 | 21.64 | 3.29 | 10 | 22.79 | 8.78 | 10 | 22.36 | 6.73 |
| | Gemcitabine | 80 mg/kg | Q3d × 4 (Day | Subcutaneous + | | | | | | | | | |
| | | | 1, 4, 7, 10) | Intravenous | | | | | | | | | |
| 7 | Oxaliplatin + | 10 mg/kg + | Day 1 + Q3d × | Intravenous + | 3 | 23.67 | 15.00 | 3 | 23.73 | 15.32 | 3 | 23.90 | 16.13 |
| | Gemcitabine | 80 mg/kg | 3 (Day 1, 4, 7) | Intravenous | | | | | | | | | |
| 8 | ODSH + | 24 mg/kg + | BID × 8 days + | Intravenous + | 0 | 0 | 0 | 0 | | | 0 | | |
| | Oxaliplatin + | 10 mg/kg + | Day 1 + | Intravenous + | | | | | | | | | |
| | Gemcitabine | 80 mg/kg | Q3d × 3 | Intraperitoneal | | | | | | | | | |
| | | | (Day 1, 4, 7) | | | | | | | | | | |

\hat{ }Beginning Day 26, groups 3 and 5 were taken off initial dosing regimen and the following dosing regimen was initiated:
Gr 3: Gemcitabine (80 mg/kg IP, Q3d × 4 starting Day 26) + Nab-paclitaxel (15 mg/kg IV, 2 × weekly starting Day 26)
Gr 5: ODSH (24 mg/kg, IV BID) + Gemcitabine (80 mg/kg IP, Q3d × 4 starting Day 26) + Nab-paclitaxel (15 mg/kg IV, 2 × weekly starting day 26)
*ODSH dosed intravenously Days 1-11; dosed subcutaneously Days 12-end

TABLE 13

| | | | | | Day 22 | | | Day 26 | | |
|---|---|---|---|---|---|---|---|---|---|---|
| | | | | | | | % | | | % |
| | | | | | | Average | Weight | | Average | Weight |
| | | | | | # | Weight | Loss or | # | Weight | Loss or |
| Group | Compound | Dosage | Frequency | Dose Route* | Mice | (g) | Gain | Mice | (g) | Gain |
| 1 | Vehicle | 0 mg/kg | BID to end | Intravenous/ | 10 | 21.49 | 3.02 | 10 | 21.46 | 2.88 |
| | Control | | | Subcutaneous | | | | | | |
| 2 | ODSH | 24 mg/kg | BID to end | Intravenous/ | 10 | 22.53 | 4.60 | 10 | 22.46 | 4.27 |
| | | | | Subcutaneous | | | | | | |
| 3\hat{ } | Oxaliplatin | 10 mg/kg | Wkly × 4 | Intravenous | 0 | 0 | 0 | 0 | | |
| | Gemcitabine | 80 mg/kg | (Day 1, 8, 15, 22) | Intraperitoneal | | | | | | |
| | Nab-paclitaxel | 15 mg/kg | Day 26, 29, 32 | Intravenous | | | | | | |
| | | | Day 26, 29, 32 | | | | | | | |
| 4 | Gemcitabine | 80 mg/kg | Q3d × 4 (Day 1, 4, 7, 10) | Intraperitoneal | 10 | 24.41 | 8.59 | 10 | 24.21 | 7.70 |
| 5\hat{ } | ODSH + | 24 mg/kg + | BID to end + Wkly × 4 | Intravenous/ | 1 | 19.10 | −9.52 | 1 | 17.20 | −18.52 |
| | Oxaliplatin | 10 mg/kg | (Day 1, 8, 15, 22) | Subcutaneous + | | | | | | |
| | Gemcitabine | 80 mg/kg | Day 26, 29, 32, 35 | Intravenous | | | | | | |
| | Nab-paclitaxel | 15 mg/kg | Day 26, 29, 32 | Intraperitoneal | | | | | | |
| | | | | Intravenous | | | | | | |
| 6 | ODSH + | 24 mg/kg + | BID to end + Q3d × 4 | Intravenous/ | 10 | 22.29 | 6.40 | 10 | 22.03 | 5.16 |
| | Gemcitabine | 80 mg/kg | (Day 1, 4, 7, 10) | Subcutaneous + | | | | | | |
| | | | | Intravenous | | | | | | |
| 7 | Oxaliplatin + | 10 mg/kg + | Day 1 + Q3d × 3 | Intravenous + | 3 | 23.87 | 15.97 | 3 | 24.37 | 18.40 |
| | Gemcitabine | 80 mg/kg | (Day 1, 4, 7) | Intravenous | | | | | | |
| 8 | ODSH + | 24 mg/kg + | BID × 8 days + | Intravenous + | 0 | | | 0 | | |
| | Oxaliplatin + | 10 mg/kg + | Day 1 + | Intravenous + | | | | | | |
| | Gemcitabine | 80 mg/kg | Q3d × 3 (Day 1, 4, 7) | Intraperitoneal | | | | | | |

\hat{ }Beginning Day 26, groups 3 and 5 were taken off initial dosing regimen and the following dosing regimen was initiated:
Gr 3: Gemcitabine (80 mg/kg IP, Q3d × 4 starting Day 26) + Nab-paclitaxel (15 mg/kg IV, 2 × weekly starting Day 26)
Gr 5: ODSH (24 mg/kg, IV BID) + Gemcitabine (80 mg/kg IP, Q3d × 4 starting Day 26) + Nab-paclitaxel (15 mg/kg IV, 2 × weekly starting day 26)
*ODSH dosed intravenously Days 1-11; dosed subcutaneously Days 12-end

Efficacy was assessed by comparison of tumor weights at Day 26 and 36 against Day 1. Day 26 was chosen to assess data prior to the addition of gemcitabine and nab-paclitaxel to groups 3 and 5. Day 36 was assessed as the last day of the study.

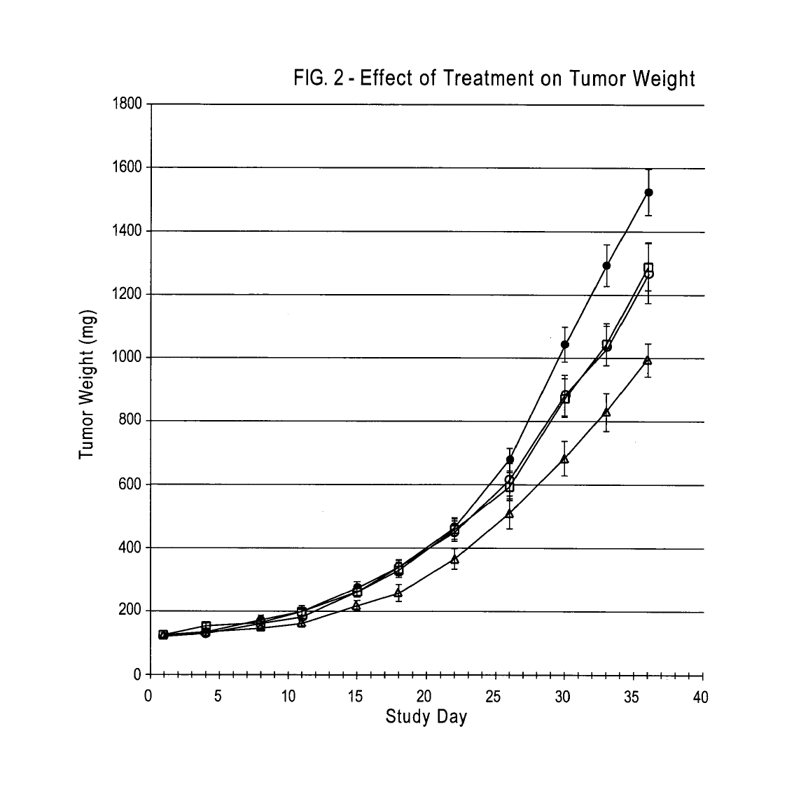

Tables 14 and 15, below, show the tumor weight and percent tumor growth inhibition (% TGI) for all treatment groups relative to Group 1 (the vehicle control group) at Day 26 and Day 36. See also FIG. 1 and FIG. 2.

TABLE 14

| | | | | | Day 26 | | |
|---|---|---|---|---|---|---|---|
| | | | | | Tumor | Day 26 | |
| Group | Treatment¹ | N | Dose | Schedule | Weight (mg) | % TGI (n) | Deaths² |
| 1 | Vehicle | 10 | — | BID to end | 679.8 ± 35.4 | — | 0 |
| 2 | ODSH | 10 | 24 mg/kg | BID to end | 616.2 ± 50.9 | 11.5 (10/10) | 0 |
| 3 | Oxaliplatin | 10 | 10 mg/kg | Wkly × 4 | 635.0 ± 52.3 | 8.0 (10/10) | 0 |
| 4 | Gemcitabine | 10 | 80 mg/kg | Q3D × 4 | 594.7 ± 43.8 | 15.3 (10/10) | 0 |
| 5 | ODSH | 10 | 24 mg/kg | BID to end | 586.6 ± 48.9 | 16.7 (10/10) | 0 |
| | Oxaliplatin | | 10 mg/kg | Wkly × 4 | | | |
| 6 | ODSH | 10 | 24 mg/kg | BID to end | 508.9 ± 47.9 | 30.7 (10/10) | 0 |
| | Gemcitabine | | 80 mg/kg | Q3D × 4 | | | |
| 7 | Oxaliplatin | 10 | 10 mg/kg | Day 1 | 612.4 ± 94.3 | 15.5 (3/10) | 7 |
| | Gemcitabine | | 80 mg/kg | Q3D × 3 | | | |
| 8 | ODSH | 10 | 24 mg/kg | BID to end | — | — | 10 |
| | Oxaliplatin | | 10 mg/kg | Day 1 | | | |
| | Gemcitabine | | 80 mg/kg | Q3D × 3 | | | |

¹Treatment administered until Day 26
²Total deaths on or before Day 26

TABLE 15

| | | | | | Day 36 | | |
|---|---|---|---|---|---|---|---|
| | | | | | Tumor | Day 36 | |
| | | | | | Weight | % TGI | |
| Group | Treatment¹ | n | Dose | Schedule | (mg) | (n) | Deaths² |
| 1 | Vehicle | 10 | — | BID to end | 1524.2 ± 72.3 | — | 0 |
| 2 | ODSH | 10 | 24 mg/kg | BID to end | 1270.1 ± 95.5 | 18.2 (10/10) | 0 |
| 3 | Oxaliplatin | 10 | 10 mg/kg | 2 × wkly × 4 | — | — | 10 |
| | Gemcitabine | | 80 mg/kg | Day | | | |
| | nab-paclitaxel | | 15 mg/kg | 26, 29, 32 | | | |
| | | | | Day | | | |
| | | | | 26, 29, 32 | | | |
| 4 | Gemcitabine | 10 | 80 mg/kg | Q3D × 4 | 1288.8 ± 74.3 | 16.8 (10/10) | 0 |
| 5 | ODSH | 10 | 24 mg/kg | BID to end | 827.5 (n = 1) | 51.4 (1/10) | 9 |
| | Oxaliplatin | | 10 mg/kg | 1 × Wkly × 4 | | | |
| | Gemcitabine | | 80 mg/kg | Day | | | |
| | nab-paclitaxel | | 15 mg/kg | 26, 29, 32, 35 | | | |
| | | | | Day | | | |
| | | | | 26, 29, 32 | | | |
| 6 | ODSH | 10 | 24 mg/kg | BID to end | 993.9 ± 52.0 | 37.9 (10/10) | 0 |
| | Gemcitabine | | 80 mg/kg | Q3D × 4 | | | |
| 7 | Oxaliplatin | 10 | 10 mg/kg | Day 1 | 1227.7 ± 244.7 | 22.5 (3/10) | 7 |
| | Gemcitabine | | 80 mg/kg | Day 1, 4, 7 | | | |
| 8 | ODSH | 10 | 24 mg/kg | BID to end | — | — | 10 |
| | Oxaliplatin | | 10 mg/kg | Day 1 | | | |
| | Gemcitabine | | 80 mg/kg | Day 1, 4, 7 | | | |

¹Treatment administered until Day 35
²Total deaths on or before Day 36

The vehicle control group (Group 1) reached a mean tumor weight of 679.8 mg by Day 26 and 1524.2 mg by Day 36. Six of ten tumors demonstrated some level of necrosis; however, this is attributed to the normal progression of this tumor xenograft model. Tumor necrosis was first observed on Day 30. A maximum weight loss of 3.6% was observed at Day 15. The mice recovered their weight by Day 26. Two of ten mice demonstrated slightly bruised tails, first observed on Day 11.

ODSH 24 mg/kg (Group 2) reached a mean tumor weight of 616.2 mg by Day 26 and 1270.1 mg by Day 36. This treatment resulted in a TGI of 11.5% on Day 26 and 18.2% on Day 36, when compared to vehicle control. No significant difference in tumor weight was observed on Day 26 or Day 36 when compared to vehicle control. Three of ten tumors demonstrated some level of necrosis; however, this is attributed to the normal progression of this tumor xenograft model. Tumor necrosis was first observed on Day 30. A maximum weight loss of 2.7% was reached on Day 11. The mice recovered their weight by Day 22. All ten mice in this group demonstrated bruising on the tails or abdomen, at the site of injection. This was first observed on Day 8 for the tails and Day 15 for the abdomens. One of the ten mice also demonstrated swelling of the tail, first observed on Day 11.

Oxaliplatin 10 mg/kg or gemcitabine 80 mg/kg, and nab-paclitaxel 15 mg/kg (Group 3): The initial regimen of oxaliplatin alone reached a mean tumor weight of 635.0 mg by Day 26, prior to the addition of gemcitabine and nab-paclitaxel to the dosing regimen. This group produced a TGI of 8.0% on Day 26, when compared to vehicle control. No significant difference in tumor weight on Day 26 was observed when compared to vehicle control. One mouse exhibited a bruised tail, first observed on Day 11. Three of ten tumors demonstrated some level of necrosis; however, this is attributed to the normal progression of this tumor xenograft model. Tumor necrosis was first observed on Day 26.

Following data collection on Day 26, the combination treatment regimen of gemcitabine and nab-paclitaxel was initiated. This regimen proved to be toxic following the initial oxaliplatin alone treatment. No efficacy data could be reported for the triple combination.

Gemcitabine 80 mg/kg (Group 4) reached a mean tumor weight of 594.7 mg by Day 26 and 1288.8 mg by Day 36. This treatment resulted in a TGI of 15.3% on Day 26 and 16.8% on Day 36, when compared to vehicle control. No significant difference in tumor weight was observed on Day 26 or Day 36 when compared to vehicle control. Four of the ten tumors demonstrated some level of necrosis; however, this is attributed to the normal progression of this tumor xenograft model. Tumor necrosis was first observed on Day 26. A maximum weight loss of 8.7% was reached on Day 11. The mice recovered their weight by Day 18.

ODSH 24 mg/kg and oxaliplatin 10 mg/kg or ODSH 24 mg/kg, gemcitabine 80 mg/kg, and nab-paclitaxel 15 mg/kg (Group 5): The initial treatment combination of ODSH and oxaliplatin reached a mean tumor weight of 586.6 mg by Day 26. This treatment resulted in a TGI of 16.7% on Day 26 when compared to vehicle control. No significant difference in tumor weight was observed on Day 26 when compared to vehicle control, ODSH (Group 2), or oxaliplatin (Group 3). All ten mice in this group demonstrated increased bruising on the tails or abdomen, at the site of injection. This was first observed on Day 4 for the tails and Day 15 for the abdomens. Two of the ten mice also demonstrated swelling of the tail, first observed on Day 4. Three of the ten mice demonstrated some discoloration of the skin, first observed on Day 11.

The triple combination of ODSH, gemcitabine, and nab-paclitaxel, initiated on Day 26, resulted in increased toxicity following the initial treatment regimen of ODSH and oxaliplatin. No statistical analysis could be performed on Day 36 because only one mouse remained in this group to Day 36 with a tumor size of 827.5 mg (TGI=51.4%).

ODSH 24 mg/kg and gemcitabine 80 mg/kg (Group 6) reached a mean tumor weight of 508.9 mg by Day 26 and 993.9 mg by Day 36. This treatment resulted in a TGI of 30.7% on Day 26 and 37.9% on Day 36 when compared to vehicle control. No significant difference in tumor weight was observed on Day 26 when compared to vehicle control, ODSH (Group 2), or gemcitabine (Group 4). A significant decrease in tumor weight was seen on Day 36 (P<0.05) when compared to vehicle control; however, no significant difference in tumor weights resulted when compared to ODSH (Group 2) or gemcitabine (Group 4). One of ten tumors demonstrated some level of necrosis; however, this is attributed to the normal progression of this tumor xenograft model. Tumor necrosis was first observed on Day 30. A maximum weight loss of 13.4% was reached on Day 11. The mice recovered their weight by Day 22. All ten mice in this group demonstrated bruising on the tails or abdomen, at the site of injection. This was first observed on Day 4 for the tails and Day 15 for the abdomens. Two of the ten mice also demonstrated swelling of the tail, first observed on Day 4. One of ten mice demonstrated discoloration of the skin, first observed on Day 9. Two of ten mice demonstrated dry skin, first observed on Day 9.

Oxaliplatin 10 mg/kg and gemcitabine 80 mg/kg (Group 7) reached a mean tumor weight of 612.4 mg by Day 26 and 1227.7 mg by Day 36. This group produced a TGI of 15.5% on Day 26 (n=3) and 22.5% on Day 36 (n=3), when compared to the vehicle control. No significant difference in tumor weight was observed on Day 26, when compared to vehicle control, oxaliplatin (Group 3), or gemcitabine (Group 4). No significant difference in tumor weight was observed on Day 36 when compared to vehicle control or gemcitabine (Group 4). One of ten tumors demonstrated some level of necrosis; this is attributed to the natural progression of the xenograft model. Tumor necrosis was first observed on Day 30. A maximum weight loss of 16.6% was reached on Day 8. The mice recovered their weight by Day 15 following cessation of gemcitabine treatment. This treatment regimen proved to be toxic. Mice were found dead on Days 10, 11, and 14, and moribund sacrificed on Days 11 and 12. Two of ten mice in this group demonstrated bruising on the tails. This was first observed on Day 4.

ODSH 24 mg/kg, oxaliplatin 10 mg/kg, and gemcitabine 80 mg/kg (Group 8) could not be assessed for efficacy due to the toxicity of the regimen driven by the oxaliplatin and gemcitabine doses.

Treatment with ODSH alone was well-tolerated although some bruising and swelling at the site of injections occurred. Therefore, the dosing route was changed to subcutaneous injection at Day 12. The combination treatments of ODSH and gemcitabine and ODSH and oxaliplatin were tolerated. Conversely, treatment combination regimens that included gemcitabine with oxaliplatin or gemcitabine and nab-paclitaxel resulted in toxicity.

The combination of ODSH and gemcitabine resulted in the best efficacy at Day 26 and Day 36. On both comparison days, the combination of ODSH and gemcitabine resulted in notably lower tumor weights than gemcitabine alone. The tumor weights of mice treated with ODSH and gemcitabine were statistically significantly lower than tumor weights in the control (saline alone) group on Day 36. See FIG. 2.

The addition, on Day 26 of the study, of gemcitabine and nab-paclitaxel to the oxaliplatin regimen in Groups 3 and 5 demonstrated severe toxicity that led to the death of many animals. It is unclear whether these toxicities were due to the combined treatment with oxaliplatin, gemcitabine and nab-paclitaxel, or residual toxicity related to the administration of oxaliplatin.

### Example 2

### Clinical Assessment of Combined ODSH/Chemotherapy Therapy in Pancreatic Cancer

A clinical trial is conducted to confirm the therapeutic advantage of combined therapy with ODSH and chemotherapy over therapy with chemotherapy alone, in the treatment of pancreatic cancer. Subjects included in the trial are: patients diagnosed with metastatic pancreatic cancer. Subjects are randomly assigned to either a control or a treatment group, the control group receiving gemcitabine therapy (gemcitabine alone arm) and the treatment group receiving ODSH in combination with gemcitabine (ODSH/gemcitabine arm) or ODSH in combination with oxaliplatin, optionally with 5-fluorouracil. Subjects receiving ODSH are given a bolus intravenous injection of 4 mg/kg ODSH concurrently with each administration of chemotherapeutic agent, followed by continuous infusion of ODSH (0.375 mg/kg/hr) over 48 to 96 hours. Gemcitabine therapy is given as described in the prescribing information for Gemzar®. See also, Burris, H. A., et al., 1997, J. Clin. Oncol. 15(6):2403-13. For oxaliplatin regimens and 5-fluorouracil regimens, see Ghosn M, et al., 2007, Am. J. Clin. Oncol. 30(1):15-20. Subjects in each arm of the trial are evaluated for time to tumor progression, weight loss, pain control, six-month survival rates, and overall survival.

A further clinical trial is carried out to confirm the therapeutic advantage of combined therapy with ODSH and chemotherapy over chemotherapy alone in the treatment of pancreatic cancer. The trial is conducted comparing combined therapy with ODSH and gemcitabine with nab-paclitaxel to therapy with gemcitabine and nab-paclitaxel (Abraxane®, albumin-bound paclitaxel) alone. Subjects included in the trial are: patients diagnosed with metastatic pancreatic cancer. First, ten subjects are treated with nab-paclitaxel at 125 mg/m² as an intravenous infusion over 30 minutes followed by gemcitabine at 1000 mg/m² as an intravenous infusion over 30 minutes, followed immediately by ODSH at 4 mg/kg as a bolus over 5 minutes, and a further administration of ODSH, thereafter, as a continuous intravenous infusion over 48 hours, at a dose of 0.375 mg/kg/hr.

Next, 50 subjects are then randomly assigned to one of two arms: either a control or a treatment group. The control group receives nab-paclitaxel at 125 mg/m² as an intravenous infusion over 30 minutes followed by gemcitabine at 1000 mg/m² as an intravenous infusion over 30 minutes, and the treatment group receives the same nab-paclitaxel and gemcitabine regimen, followed immediately by ODSH at 4 mg/kg as a bolus over 5 minutes, and a further administration of ODSH, thereafter, as a continuous intravenous infusion over 48 hours, at a dose of 0.375 mg/kg/hr. Administration of chemotherapeutic agents with or without ODSH is carried out once a week for three weeks, followed by a week of rest. Subjects in each group (control and treatment) are evaluated for progression-free survival, incidence of adverse events and toxicity, overall survival, objective tumor response, and ODSH plasma concentration and/or area under the curve (AUC) during bolus and infusion administrations.

Results are obtained which demonstrate that addition of ODSH to chemotherapy has a therapeutic benefit in the treatment of pancreatic cancer.

### Example 3

### Clinical Assessment Of Combined ODSH/Chemotherapy in Gastric Cancer

A clinical trial is conducted to confirm the therapeutic advantage of combined therapy with ODSH and modified docetaxel, cisplatin, fluorouracil (mDCF) therapy over mDCF therapy alone, in the treatment of gastric cancer. Subjects included in the trial are: patients diagnosed with metastatic gastric, including cancer at the gastroesophageal junction. Subjects are randomly assigned to either a control or a treatment group, the control group receiving mDCF therapy and the treatment group receiving ODSH in combination with mDCF therapy (ODSH/mDCF arm). In the ODSH/mDCF arm, subjects are given a bolus intravenous injection of 4 mg/kg ODSH concurrently with each bolus administration of docetaxel and cisplatin, and a continuous infusion of ODSH (0.375 mg/kg/hr) concurrently with 5-fluorouracil infusion, followed by a further infusion of ODSH over 48 to 96 hours. mDCF therapy is described in Shah, M. A., et al., 2010, J. Clin. Oncol., 28(15) (May 20 Supplement): 4014. Subjects in each arm of the trial are evaluated for time to tumor progression, weight loss, pain control, six-month survival rates, and overall survival.

Results are obtained which demonstrate that addition of ODSH to chemotherapy has a therapeutic benefit in the treatment of gastric cancer.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of treating cancer, comprising:
adjunctively administering 2-O, 3-O desulfated heparin (ODSH) and a cancer treatment to a subject in need thereof.

2. The method of claim 1, wherein ODSH is administered parenterally.

3. The method of claim 2, wherein ODSH is administered intravenously.

4. The method of claim 3, wherein ODSH is administered as a bolus.

5. The method of claim 3, wherein ODSH is administered as an infusion.

6-12. (canceled)

13. The method of claim 1, wherein ODSH is administered at a dosage of about 1 mg/kg to about 20 mg/kg.

14. The method of claim 1, wherein ODSH is administered at a dosage of about 0.1 mg/kg/hr to about 2.5 mg/kg/hr.

15. The method of claim 1, wherein the subject has pancreatic cancer.

16-19. (canceled)

20. The method of claim 15, wherein the cancer treatment is chemotherapy.

21. The method of claim 20, wherein ODSH is administered intravenously.

22. The method of claim 21, wherein ODSH is administered in combination with gemcitabine.

23. The method of claim 21, wherein ODSH is administered in combination with nab-paclitaxel and gemcitabine.

24-100. (canceled)

101. The method according to claim 1 further comprising a step of determining expression level of RAGE or HMGB1 in a tumor sample from the subject, prior to administration of ODSH.

102. In a method of treating cancer using a tumor-appropriate cancer treatment, the improvement comprising:
adjunctively administering 2-O, 3-O desulfated heparin (ODSH) and a cancer treatment to a subject suffering from cancer.

103-104. (canceled)
